(19) United States
(12) Patent Application Publication
(10) Pub. No.: US 2020/0323888 A1
(43) Pub. Date: Oct. 15, 2020

(54) INHIBITORS

(71) Applicant: VESTLANDETS INNOVASJONSSELSKAP AS, Bergen (NO)

(72) Inventors: Thomas ARNESEN, Nesttun (NO); Bengt Erik HAUG, Tertnes (NO); Line Merethe MYKLEBUST, Bergen (NO); Havard FOYN, Spikkestad (NO)

(73) Assignee: VESTLANDETS INNOVASJONSSELSKAP AS, Bergen (NO)

(21) Appl. No.: 16/956,776

(22) PCT Filed: Dec. 19, 2018
(86) PCT No.: PCT/IB2018/060365

§ 371 Date: Jun. 22, 2020

(30) Foreign Application Priority Data
Dec. 20, 2017 (GB) 1721465.1

(51) Int. Cl. A61K 31/685; C07F 9/22; C07K 5/10; C07K 5/113; A61K 47/64; A61K 38/03; A61K 38/07

(52) U.S. Cl. CPC A61K 31/685; C07F 9/222; C07K 5/10; A61K 38/07; A61K 47/64; A61K 38/03; C07K 5/1021

(57) ABSTRACT

The present invention relates to compounds having the formula [Co-enzyme A or analogue thereof]-Z1-Z2-Z3-Z4, wherein Z1 is a linker, Z2 and Z3 are peptides or peptide-based moieties, and Z4 is a C-terminal group. The invention also provides pharmaceutical compositions comprising compounds of the invention, and their uses for the treatment of cancer, wound healing and nerve regeneration, inter alia.

The present invention relates to compounds having the formula [Co-enzyme A or analogue thereof]-Z1-Z2-Z3-Z4, wherein Z1 is a linker, Z2 and Z3 are peptides or peptide-based moieties, and Z4 is a C-terminal group. The invention also provides pharmaceutical compositions comprising compounds of the invention, and their uses for the treatment of cancer, wound healing and nerve regeneration, inter alia.

N-terminal (Nt)-acetylation plays an important role in regulating eukaryotic cell signaling. The majority of eukaryotic proteins are Nt-acetylated. The functional consequences of this modification are quite diverse, including degradation of Nt-acetylated proteins by a novel branch of the N-end rule pathway; inhibition of post-translational ER-translocation; protein-complex formation; and protein targeting to membranes.

Nt-acetylation occurs when the acetyl moiety of acetyl Co-enzyme A (Ac-CoA) is transferred to the a-amino group of a polypeptide by an N-terminal acetyltransferase (NAT). Currently, six human NATs are known, i.e. NatA-NatF. Each NAT is composed of one or more distinct subunits, and these enzymes acetylate specific subsets of proteins that are largely defined by their N-terminal amino acid sequence. For example, the highly conserved NatA complex, which is composed of the catalytic subunit hNaa10 (Ard1) and the non-enzymatic auxiliary subunit Naa15 (Nat1/NATH), acetylates N-termini starting with Ser, Ala, Thr, Gly, Val, and Cys; NatA acts on these N-termini after the initiator Met (iMet) has been removed by methionine aminopeptidases.

A number of studies have reported the up-regulated expression, at both the mRNA and protein level, of the two main components of the NatA complex (i.e. hNaa10 and hNaa15) in several different cancer types, suggesting that NatA is associated with cancer development, proliferation and survival (e.g. Kalvik, T. V. and Arnesen, T. (2013) “Protein N-terminal acetyltransferases in cancer”, Oncogene 32, 269-276; and Drazic A. et al., “The world of protein acetylation”, Biochim Biophys Acta. 2016, 1864(10): 1372-401).

Furthermore, knockdown of hNaa10 in a variety of thyroid cancer cell lines has been shown to inhibit cell proliferation and increased sensitivity to drug-induced cytotoxicity (Gromyko, D. et al. (2010) “Depletion of the human Na-terminal acetyltransferase A induces p53-dependent apoptosis and p53-independent growth inhibition”. Int. J. Cancer 127, 2777-2789).

Additionally, knockdown studies have shown that the entire NAT machinery (i.e. NatA-NatF) is important for cell proliferation and normal cell cycle progression (Kalvik supra). Kalvik also reports that NATs have been suggested to act as oncoproteins in human cancers and that NAT expression may be elevated in certain cancer tissues (see Table 1, and references therein).

A growing number of studies have reported that a high expression of hNaa10p in certain cancers seems to correlate with a low survival rate and aggressiveness of tumours (e.g. Lee CF et al. “hNaa10p contributes to tumorigenesis by facilitating DNMT1-mediated tumor suppressor gene silencing”. J. Clin. Invest. 2010; 120: 2920-2930; and Ren T, et al. “Generation of novel monoclonal antibodies and their application for detecting ARD1 expression in colorectal cancer”, Cancer Lett. 2008; 264: 83-92).

NAA10 has been found overexpressed in cancers of different types and tissues such as hepatocellular carcinoma, colorectal cancer, lung cancer and breast cancer. Over-expression of hNaa10p was also reported in the urinary bladder cancer, breast cancer and cervical carcinoma.

Knockdown of hNaAA10 and hNAA15 resulted in significant reduction of cell growth of human colon carcinoma; and knockdown of hNaAA10 suppressed proliferation of non-small lung cancer cells and thyroid carcinoma cell lines. These studies strongly support a role for Naa10 in promoting cell proliferation and cell survival. Given these results, the NATs are potential anti-cancer targets.

The synthesis of some NAT inhibitors has previously been reported (Foyn H, et al. (2013) “Design, synthesis, and kinetic characterization of protein N-terminal acetyltransferase inhibitors”. ACS chemical biology 8(6):1121-1127). In particular, bisubstrate analogues that inhibit the NatA complex (CoA-Ac-SES4 (SEQ ID NO: 26); IC₅₀=15.1 μM), hNaa10, the catalytic subunit of NatA (CoA-Ac-EEE4 (SEQ ID NO: 7); IC₅₀=10.1 μM), and NatE/hNaa50 (CoA-Ac-MLG7 (SEQ ID NO: 49); IC₅₀=1.29 μM) were disclosed, inter alia. The aforementioned NatA and Naa10 inhibitors were subsequently found to be of low selectivity.

There remains a need, however, for alternative NAT inhibitors which can be useful for inhibiting cell proliferation and cell survival, e.g. useful for the prevention or treatment of cancer.

The invention also relates to inhibitors of a further N-terminal acetyltransferase, Naa80. Whilst Naa80 (Fus-2/NAT6) has previously been described as a putative NAT (Zegerman P, et al. (2000), “The putative tumour suppressor Fus-2 is an N-acetyltransferase”, Oncogene 19(1):161-163), this was based only on sequence analysis that suggested the presence of a NAT-specific GNAT-fold. However, no specific substrate has previously been described for Naa80.

The inventors have now studied the Nt-acetylation activity of Naa80 towards a number of peptides and discovered a marked preference for peptides with acidic N-termini, such as those found in actin.

It has previously been suggested that Naa10 inhibitors based on EEE (actin N-terminus) could be of use in the treatment of cancer (Foyn H, et al. (2013) “Design, synthesis, and kinetic characterization of protein N-terminal acetyltransferase inhibitors”, ACS chemical biology 8(6):1121-1127) due to Naa10's roles in cancer. However, it has now been found that those actin-based inhibitors are not selective Naa10 inhibitors, and hence would not be useful in the treatment of cancer.

Studies described herein in cell lines in which the NAA80 gene has been knocked out have demonstrated that cells which are deficient in actin Nt-acetylation have increased motility. Hence inhibitors of Naa80 will have utility in the treatment of diseases or disorders wherein cell motility is to be enhanced, such as nerve regeneration and wound healing.

It is therefore an object of the invention to provide compounds which can be useful for inhibiting cell proliferation and cell survival, e.g. useful for the prevention or treatment of cancer, and compounds which can be useful in the treatment of diseases or disorders wherein cell motility is to be enhanced, e.g. wound healing and nerve regeneration.

In one embodiment, the invention provides a compound of Formula I:

[Co-enzyme A or analogue thereof]-Z1-Z2-Z3-Z4 (I)

wherein

Z1 is a linker moiety or is absent;

Z2 consists of a peptide or peptide-based moiety having the Formula II:

(II)
(SEQ ID NO: 1)
X1-X2-X3-X4

wherein
X1 and X2 are independently S or Hse;
X3 is M or Nle; a non-natural amino acid; a C₁₋₅ cyclic or non-cyclic alkyl
group; or a C₁₋₅ ether group;
X4 is P or is absent;

or Z2 consists of a peptide having the Formula III:

(III)
(SEQ ID NO: 2)
X5-X6-X7-X8

wherein
X5 is D, E, M or Q
X6 is D, E or S
X7 is D, E or Q
X8 is I or L;

Z3 is 0-30 amino acids; and

Z4 is a C-terminal group or is absent,

or a pharmaceutically-acceptable salt thereof.

The compound of Formula I comprises a Co-enzyme A moiety or an analogue thereof. As used herein, the term “Co-enzyme A” refers to the moiety:

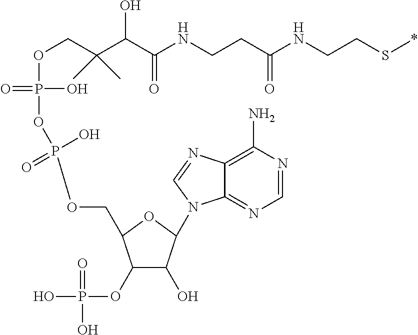 [SMILES: *SCCNC(=O)CCNC(=O)C(O)C(C)(C)COP(=O)(O)OP(=O)(O)OCC1OC(n2cnc3c(N)ncnc32)C(O)C1OP(=O)(O)O]

wherein * refers to the point of connection to Z1.

As used herein, the term “Co-enzyme A moiety or an analogue thereof” includes compounds of formula:

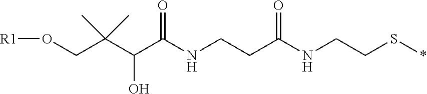

wherein R1 is hydrogen, a phosphate group, acetoacetate, alkyl, aralkyl or cyclic alkyl.

Most preferably, the analogue of Co-enzyme A is:

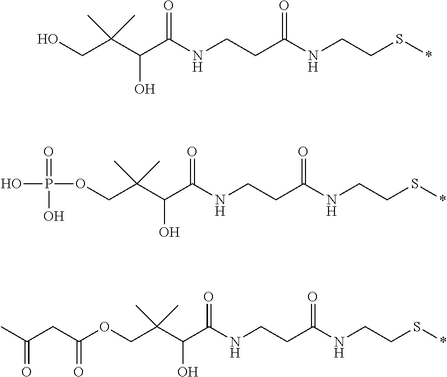 [SMILES: *SCCNC(=O)CCNC(=O)C(O)C(C)(C)CO ; *SCCNC(=O)CCNC(=O)C(O)C(C)(C)COP(=O)(O)O ; *SCCNC(=O)CCNC(=O)C(O)C(C)(C)COC(=O)CC(C)=O]

Z1 is a linker moiety or is absent. The term “linker moiety” refers to a linear or cyclic chemical moiety (which may or may not be substituted) which provides attachment of the Co-enzyme A moiety or analogue thereof to Z2. Examples of suitable linker moieties include a linear peptide consisting of 1-10 amino acids (e.g. -Gly)ₙ- and -(CH₂)ₙ-, where n is 1-10. Preferably Z1 is an acetyl group (i.e. —CH₂—C(O)—), or a variant or derivative thereof.

More preferably, Co-enzyme A-Z1 forms the compound:

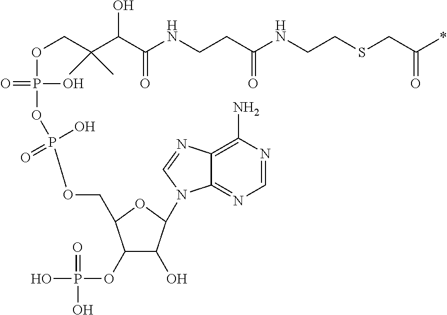 [SMILES: *C(=O)CSCCNC(=O)CCNC(=O)C(O)C(C)(C)COP(=O)(O)OP(=O)(O)OCC1OC(n2cnc3c(N)ncnc32)C(O)C1OP(=O)(O)O]

wherein * indicates the point of connection to Z2.

In one embodiment, Z2 consists of a peptide or peptide-based moiety having the

Formula II:

(SEQ ID NO: 1)
X1-X2-X3-X4 (II)

wherein
X1 and X2 are independently S or Hse;
X3 is M or Nle; a non-natural amino acid; a C₁₋₅ cyclic or non-cyclic alkyl
group; or a C₁₋₅ ether group; and
X4 is P or is absent.

Preferably, X1-X2-X3-X4 has the amino acid sequence:

(SEQ ID NO: 3)
SSMP or
(SEQ ID NO: 4)
SS(NLe)P.

In the above peptide sequences, the amino acids are represented by their standard one- or three-letter codes (i.e. S=serine, M=methionine, P=proline, Nle=norleucine and Hse=homo-serine).

Preferably, the non-natural amino acid is 2-amino-4-cyclopropylbutanoic acid, 2-amino-3-cyclopropylpropanoic acid or 2-amino-2-cyclohexylacetic acid.

In a further embodiment, Z2 consists of a peptide of Formula III, wherein the amino acid sequence of the peptide is

(SEQ ID NO: 2)
X5-X6-X7-X8 (III)

wherein
X5 is D, E, M or Q
X6 is D, E or S
X7 is D, E or Q
X8 is I or L;

In the above peptide sequences, the amino acids are represented by their standard one-letter codes (i.e. D=aspartic acid, E=glutamic acid, M=methionine, P=proline, S=serine, Q=glutamine, I=isoleucine and L=leucine).

Preferably, Z2 consists of a peptide, wherein the amino acid sequence of the peptide is

(SEQ ID NO: 5)
X5-X6-X7-X8

wherein
X5 is E or D
X6 is D or E
X7 is D or E, and
X8 is I or L.

More preferably, Z2 consists of a peptide, wherein the amino acid sequence of the peptide is selected from the group consisting of:

(SEQ ID NO: 6)
DDDI,
(SEQ ID NO: 7)
EEEI,
(SEQ ID NO: 8)
MDEL,
(SEQ ID NO: 9)
DEDI,
(SEQ ID NO: 10)
DEEL,
(SEQ ID NO: 11)
EDDI,
(SEQ ID NO: 12)
EDEI,
(SEQ ID NO: 13)
EEDL,
(SEQ ID NO: 14)
EEEL,
(SEQ ID NO: 15)
DDEI,
(SEQ ID NO: 16)
EDQL,
(SEQ ID NO: 17)
ESEL,
(SEQ ID NO: 18)
DEEI,
(SEQ ID NO: 19)
EEDI,
(SEQ ID NO: 20)
EDEL.
(SEQ ID NO: 21)
QEEI

In some particularly preferred embodiments, Z2 consists of a peptide, wherein the amino acid sequence of the peptide is selected from the group consisting of:

(SEQ ID NO: 6)
DDDI,
(SEQ ID NO: 7)
EEEI,
(SEQ ID NO: 11)
EDDI,
(SEQ ID NO: 19)
EEDI or
(SEQ ID NO: 20)
EDEL.

Z3 is 0-30 amino acids, preferably 1-8 amino acids. For example, Z3 may be 0, 1, 2, 3, 4, 5, 6, 7, 8, 9 or 10 amino acids.

In some embodiments, Z3 is selected from 1-10 contiguous amino acids obtained from the sequence: (A/T)ALV(V/I/C)DNGSG (SEQ ID NO: 22). More preferably, Z3 is absent (i.e. a single bond connects Z2 to Z4).

In some embodiments, Z3 comprises 1-12 arginine (R) amino acids. Z3 may constitute a linear or cyclic structure. Such amino acids may aid cell penetration.

In some embodiments, Z3 comprises or consists of K (lysine). In some preferred embodiments, Z2-Z3 is SS(Nle)PK (SEQ ID NO: 23).

Z4 is a C-terminal group or is absent. The C-terminus of the amino acid sequence in Z2-Z3 may be a substituted or unsubstituted amide group. For example, the amide group may be substituted to form an N-alkyl or N,N-dialkyl amide (wherein alkyl is preferably a C₁₋₅ alkyl chain).

Primary and secondary amide groups are preferred. Suitable groups to substitute the amide group include amino acids, e.g. lysine or ornithine; aminoalkyl, e.g. amino ethyl or dimethylaminoethyl; the nitrogen atom of the amide group may form part of a cyclic group, e.g. pyrazolidine, piperidine, imidazolidine and piperazine, with piperazine being preferred. These cyclic groups may themselves be substituted, for example by alkyl or aminoalkyl groups. Preferably, Z4 is —NH₂ or N-alkyl.

Preferably, the amino acids present in Z1-Z3 are L-amino acids. In some embodiments, one or more of these amino acids may be D-amino acids. In particular, X3 and/or one or more of the Z3 amino acids may be a D-amino acid.

A targeting moiety may be attached to the compound of Formula I at any suitable location. The targeting moiety may be one which targets the compound of Formula I to a desired site, e.g. to a particular organ, tissue or cell-type. For example, the targeting moiety may target the compound of Formula I to a cancer cell, e.g. wherein the cancer is selected from the group consisting of lung cancer, breast cancer, hepatocellular carcinoma, colorectal cancer, pancreatic cancer, prostate cancer, ovarian cancer, gastric cancer, non-small cell lung cancer, papillary thyroid carcinoma, neuroblastoma and thyroid cancer.

Preferably, the targeting moiety is attached to one or more of Z2, Z3 or Z4; more preferably, it is attached to Z3.

Examples of targeting moieties include peptides (cyclic or linear) and non-peptide groups, e.g. folic acid. Preferred targeting moieties include peptides and peptidomimetics and non-peptide groups which bind specifically to a prostate-specific membrane antigen, somatostatin receptor, integrin receptor or folic acid receptor. The term “targeting moiety” also includes cell-penetrating peptides and analogues thereof. Particularly preferred targeting moieties include c(RGDyK) (SEQ ID NO: 24), octreotide and analogues thereof.

The targeting moiety may be attached to the compound of Formula I via a cleavable or non-cleavable linker. Suitable linkers are well known in the art.

In some embodiments, the targeting moiety is CPP9 (i.e. cyclo(Phe-2-Nal-Arg-Arg-Arg-Arq-Gln (SEQ ID NO: 25), wherein the underlined residues are D-amino acids, and 2-Nal is 2-napthylalanine; see also Ziqing Qian et al, Biochemistry, 2016, 55 (18), 2601-2612). This may be linked to a K residue (in Z3 or Z4) via the alpha amino group.

In some preferred embodiments, Z2-Z3 is SS(Nle)PK-[cyclic peptide] or SS(Nle)PK-folate (SEQ ID NO: 23).

One or more subunits of the peptide moieties of the compounds of the invention may be replaced by peptidomimetic subunits. A peptidomimetic is typically characterised by retaining the polarity, three dimensional size and functionality (bioactivity) of its parent peptide, but wherein the peptide bonds have been replaced, often by more stable linkages. By ‘stable’ is meant more resistant to enzymatic degradation by hydrolytic enzymes. Generally, the bond which replaces the amide bond (amide bond surrogate) conserves many of the properties of the amide bond, e.g. conformation, steric bulk, electrostatic character, possibility for hydrogen bonding etc. (Chapter 14 of “Drug Design and Development”, Krogsgaard, Larsen, Liljefors and Madsen (Eds) 1996, Horwood Acad. Pub. provides a general discussion of techniques for the design and synthesis of peptidomimetics.)

The peptidomimetic subunits will be approximately equivalent in size and function to the amino acids which they replace. Peptidomimetics will generally have groups which are equivalent to the side-groups of the amino acids which they replace. Peptidomimetic molecules will include the same number of subunits as the parent peptide, but these subunits may typically be linked by amide bond mimics.

Preferred peptide bond mimics include esters, polyamines and derivatives thereof, as well as substituted alkanes and alkenes, particularly aminomethyl and ketomethylene.

As well as replacement of amide bonds, peptidomimetics may involve the replacement of larger structural moieties with di- or tri-peptidomimetic structures and in this case, mimetic moieties involving the peptide bond, such as azole-derived mimetics, may be used as dipeptide replacements.

The peptidomimetics will preferably have C-termini which may be modified as discussed herein and may also carry targeting groups as discussed herein.

Other peptidomimetics include peptoids formed, for example, by the stepwise synthesis of amide-functionalised polyglycines. Some peptidomimetic backbones will be readily available from their peptide precursors, such as peptides which have been permethylated, suitable methods are described by Ostresh, J. M. et al. in Proc. Natl. Acad. Sci. USA(1994) 91, 11138-11142. Strongly basic conditions will favour N-methylation over O-methylation and result in methylation of some or all of the nitrogen atoms in the peptide bonds and the N-terminal nitrogen.

The invention also relates to pharmaceutically-acceptable salts of the compounds of the invention. A “pharmaceutically-acceptable salt” refers to a salt that retains the desired biological activity of the parent compound and does not impart any undesired toxicological effects

Examples of such salts include acid addition salts and base addition salts. Acid addition salts include those derived from non-toxic inorganic acids, such as hydrochloric, nitric, phosphoric, sulphuric, hydrobromic, hydroiodic, phosphorous and the like, as well as from non-toxic organic acids such as aliphatic mono- and dicarboxylic acids, phenyl-substituted alkanoic acids, hydroxy alkanoic acids, aromatic acids, aliphatic and aromatic sulfonic acids, and the like.

Base addition salts include those derived from alkaline earth metals, such as sodium, potassium, magnesium, calcium and the like, as well as from non-toxic organic amines, such as N,N′-dibenzylethylenediamine, N-methylglucamine, chloroprocaine, choline, diethanolamine, ethylenediamine, procaine, and the like.

The molecules described herein may be synthesised in any convenient way. Generally the reactive groups present (for example amino, thiol and/or carboxyl) will be protected during overall synthesis. The final step in the synthesis of the peptide part is generally deprotection. The final step for inhibitor synthesis is generally the conjugation of Co-enzyme A to the bromo-acetyl peptide. In building up the peptide, one can in principle start either at the C-terminal or the N-terminal, although the C-terminal starting procedure is preferred. Methods of peptide synthesis are well known in the art, but for the present invention it may be particularly convenient to carry out the synthesis on a solid phase support, such supports being well known in the art.

The invention also provides a pharmaceutical composition comprising a compound of the invention, optionally together with one or more diluent, carrier or excipient. Such formulations may be for, inter alia, pharmaceutical (including veterinary) purposes and thus a suitable diluent, carrier or excipient will preferably be pharmaceutically acceptable. Suitable diluents, excipients and carriers are known to the skilled man.

As used herein, “pharmaceutically acceptable carrier” includes any and all solvents, dispersion media, coatings, antibacterial and antifungal agents, isotonic and absorption delaying agents, and the like that are physiologically compatible. Preferably, the carrier is suitable for intravenous, intramuscular, subcutaneous, parenteral, spinal or epidermal administration (e.g. by injection or infusion). Depending on the route of administration, the active compound may be coated in a material to protect the compound from the action of acids and other natural conditions that may inactivate the compound.

Examples of suitable aqueous and non-aqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of presence of micro-organisms may be ensured both by sterilization procedures, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of an injectable pharmaceutical form may be brought about by the inclusion of agents which delay absorption such as aluminium monostearate and gelatine.

Injection solutions may, for example, be produced in the conventional manner, such as by the addition of preservation agents, such as p-hydroxybenzoates, or stabilizers, such as EDTA. The solutions are then filled into injection vials or ampoules.

The amount of active ingredient which can be combined with a carrier material to produce a single dosage form will vary depending upon the subject being treated, and the particular mode of administration. The amount of active ingredient which can be combined with a carrier material to produce a single dosage form will generally be that amount of the composition which produces a therapeutic effect. Generally, out of one hundred per cent, this amount will range from about 0.01 per cent to about ninety-nine percent of active ingredient, preferably from about 0.1 per cent to about 70 per cent, most preferably from about 1 per cent to about 30 per cent of active ingredient in combination with a pharmaceutically acceptable carrier.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

For administration of the compound of the invention, the dosage ranges from about 0.0001 to 100 mg/kg, and more usually 0.01 to 5 mg/kg, of the host body weight. For example dosages can be 0.3 mg/kg body weight, 1 mg/kg body weight, 3 mg/kg body weight, 5 mg/kg body weight or 10 mg/kg body weight or within the range of 1-10 mg/kg. An exemplary treatment regime entails administration once per week, once every two weeks, once every three weeks, once every four weeks, once a month, once every 3 months or once every three to 6 months.

In employing such compositions systemically, the compound of the invention is preferably present in an amount such as to achieve a serum level of the compound of at least about 5 μg/ml. In general, the serum level need not exceed 500 μg/ml. A preferred serum level is about 100 μg/ml. Such serum levels may be achieved by incorporating the compound in a composition to be administered systemically at a dose of from 1 to about 10 mg/kg. In general, the compound need not be administered at a dose exceeding 100 mg/kg.

Preferred dosage regimens for a compound of the invention include 1 mg/kg body weight or 3 mg/kg body weight via intravenous administration, with the compound being given using one of the following dosing schedules: (i) every four weeks for six dosages, then every three months; (ii) every three weeks; (iii) 3 mg/kg body weight once followed by 1 mg/kg body weight every three weeks.

The compositions according to the invention may be presented, for example, in a form suitable for oral, topical, nasal, parenteral, intravenal, intratumoral, rectal or regional (e.g. isolated limb perfusion) administration. Administration is typically by a parenteral route, preferably by injection subcutaneously, intramuscularly, intracapsularly, intraspinally, intratumourally or intravenously.

The active compounds defined herein may be presented in the conventional pharmacological forms of administration, such as tablets, coated tablets, nasal sprays, solutions, emulsions, liposomes, nanoparticles, powders, capsules or sustained release forms. Liposomes and nanoparticles comprising compounds of Formula I are particularly preferred. Conventional pharmaceutical excipients as well as the usual methods of production may be employed for the preparation of these forms.

Preferred formulations are those in which the peptides are dissolved in saline. Such formulations are suitable for use in preferred methods of administration, especially local administration, e.g. intra-tumoural, e.g. by injection or by perfusion/infusion of a preferably isolated (including partial isolation) limb, body region or organ.

The compounds of the invention also can be administered in combination therapy, i.e., combined with other agents. For example, the combination therapy may include an compound of the invention combined with at least one other anti-tumour agent, or an anti-inflammatory or immunosuppressant agent.

Other anti-tumour agents may include different types of cytokines, e.g. IFN-γ, TNF, CSF and growth factors, immunomodulators, chemotherapeutics, e.g. cisplatin or antibodies or anti-cancer vaccines.

The subject will typically be a human subject, but non-human animals, such as domestic or livestock animals (including horses) may also be treated.

The compounds of the invention wherein Z2 is SEQ ID NO: 1 are capable of acting as inhibitors of NatA (Naa15-Naa10). NatA is involved in cell proliferation.

The invention therefore provides a compound of the invention wherein Z2 has the sequence of SEQ ID NO: 1 for use as a medicament or for use in therapy.

In a further aspect, the present invention provides a method of treating or preventing a disease or disorder associated with NatA activity, the method comprising administering an effective amount of a compound of the invention wherein Z2 has the sequence of SEQ ID NO: 1 to a subject in need thereof. The invention also provides the use of a compound of the invention wherein Z2 has the sequence of SEQ ID NO: 1 in the manufacture of a medicament for treating or preventing a disease or disorder associated with NatA activity. The invention also provides a compound of the invention wherein Z2 has the sequence of SEQ ID NO: 1 for use in treating or preventing a disease or disorder associated with NatA activity.

The disease or disorder associated with NatA activity is preferably one which is susceptible to treatment or prevention by blocking NatA activity.

In a further aspect, the present invention provides a method of treating or preventing cancer, the method comprising administering an effective amount of a compound of the invention wherein Z2 has the sequence of SEQ ID NO: 1 to a subject in need thereof. The invention also provides the use of a compound of the invention wherein Z2 has the sequence of SEQ ID NO: 1 in the manufacture of a medicament for treating or preventing cancer. The invention also provides a compound of the invention wherein Z2 has the sequence of SEQ ID NO: 1 for use in treating or preventing cancer.

The cancer is preferably one which is susceptible to treatment or prevention by blocking NatA activity. The treatment or prevention of the cancer is preferably by blocking NatA activity. Preferably, the disease is human cancer (i.e. the subject is a human). Preferably, the treating or preventing the cancer comprises treating tumour cells or preventing or reducing the growth, establishment spread, or metastasis of a tumour.

Preferred cancers include lymphomas, leukaemias, neuroblastomas and glioblastomas (e.g. from the brain), carcinomas and adenocarcinomas (particularly from the breast, colon, kidney, liver, lung, ovary, pancreas, prostate and skin) and melanomas. Most preferably, with regard to compounds of the invention wherein Z2 has the sequence of SEQ ID NO: 1, the cancer is selected from the group consisting of lung cancer, breast cancer, hepatocellular carcinoma, colorectal cancer, pancreatic cancer, ovarian cancer, gastric cancer, non-small cell lung cancer, papillary thyroid carcinoma, neuroblastoma, prostate cancer and thyroid cancer. In some embodiments, the cancer is not breast cancer.

The compounds of the invention wherein the amino acid sequence of Z2 is SEQ ID NO: 2 are capable of acting as inhibitors of Naa80. Inhibitors of Naa80 are shown herein to alter cytoskeletal dynamics, enhance cell mobility, and increase cell migration, inter alia.

The invention therefore provides a compound of the invention wherein the amino acid sequence of Z2 is SEQ ID NO: 2 for use as a medicament or for use in therapy.

In a further aspect, the present invention provides a method of treating or preventing a disease or disorder associated with Naa80 activity, the method comprising administering an effective amount of a compound of the invention wherein the amino acid sequence of Z2 is SEQ ID NO: 2 to a subject in need thereof. The invention also provides the use of a compound of the invention wherein the amino acid sequence of Z2 is SEQ ID NO: 2 in the manufacture of a medicament for treating or preventing a disease or disorder associated with Naa80 activity. The invention also provides a compound of the invention wherein the amino acid sequence of Z2 is SEQ ID NO: 2 for use in treating or preventing a disease or disorder associated with Naa80 activity.

The disease or disorder associated with Naa80 activity is preferably one which is susceptible to treatment or prevention by blocking Naa80 activity.

In a further aspect, the present invention provides a method of enhancing cell mobility or wound healing, the method comprising administering an effective amount of a compound of the invention wherein the amino acid sequence of Z2 is SEQ ID NO: 2 to a subject in need thereof. The invention also provides the use of a compound of the invention wherein the amino acid sequence of Z2 is SEQ ID NO: 2 in the manufacture of a medicament for enhancing cell mobility or for wound healing. The invention also provides a compound of the invention wherein the amino acid sequence of Z2 is SEQ ID NO: 2 for enhancing cell mobility or wound healing.

Similarly, mutatis mutandis, compounds of the invention wherein the amino acid sequence of Z2 is SEQ ID NO: 2 may be useful:

(a) in the treatment of diseases or disorders which require or which would be aided by enhanced cell mobility;

(b) for the promotion of cell mobility, cell motility or promoting cell migration;

(c) in the treatment of diseases or disorders associated with actin polymerisation or depolymerisation, or with cytoskeletal defects;

(d) for the promotion of wound healing or muscle healing, or for the treatment or prevention of Duschenne Muscular Dystrophy (DMD); and

(e) for the promotion of nerve cell migration, regeneration or reinervation.

The disclosure of each reference set forth herein is specifically incorporated herein by reference in its entirety.

### BRIEF DESCRIPTION OF THE FIGURES

FIG. 1: Immunoprecipitated Naa80-V5 was used in Nt-acetylation assays with [¹⁴C]-Ac-CoA and peptides representing a variety of N-termini.

FIG. 2: Sequence alignment (first 61-63 amino acids) of the six human mature actin isoforms generated as in (A). Of note, methionines and/or cysteines at the second position are removed prior to Nt-acetylation (arrows) of newly-formed acidic N-termini.

FIG. 3: Naa80-eGFP expressed in HeLa and HAP1 cells shows a cytosolic distribution.

FIG. 4: NAA80 KO1 cells were generated by deleting 17 bp in exon 2 and NAA80 KO2 by inserting 404 bp into exon 2 of the NAA80 gene. Putative gene product of NAA80 KO1 consists of 193 aa and in NAA80 KO2 of 151 aa protein. Both knockout gene products lack the crucial Ac-CoA binding motif.

FIG. 5: Phosphorescence imaging shows a distinct 43-kDa band in NAA80 KO1 cell lysates incubated with [¹⁴C]-Ac-CoA and purified recombinant MBP-Naa80, suggesting the incorporation of [¹⁴C]-Ac at the N-terminus of actin.

FIG. 6A: The isoform- and Nt-acetylation specificity of anti Ac-β-actin and Ac-γ-actin antibodies is confirmed in dot blot assays with peptides representing the unacetylated and acetylated N-termini of β- and γ-actin.

FIG. 6B: Naa80 expression is required for actin Nt-acetylation. Immunoblot analysis of HAP1 control and NAA80 KO1 cells transfected with either empty V5 plasmid, wild-type Naa80-V5 or the catalytically inactive mutant Naa80mut-V5. The samples were probed with anti Ac-β-actin, Ac-γ-actin, pan-actin, V5 and GAPDH antibodies.

FIG. 7: Actin Nt-acetylation affects cell motility. (A) Representative images of HAP1 control and NAA80 KO1 cells in wound-healing assay showing the degree of gap closure after 18 h. Scalebar, 150 μm. (B) Quantification of gap size in wound-healing assays relative to the size at t0. (C) Average cell front velocity (μm/h) calculated from (B). (D) Chemotaxis assay showing percentage of cells migrated from a 1% to 10% FBS chamber. (E) Random migration assay showing the percentage of cells migrating from a 10% to 10% FBS chamber. Asterisks represent significance determined by two-sided Student's t-test: *** p≤0.001.

FIG. 8: Effects of actin Nt-acetylation on cytoskeletal morphology. (A) and (B) Representative micrographs illustrating a differential morphological phenotype at the level of filopodia between HAP1 ctrl (A) and NAA80 KO1 (B) cells where the absence of Naa80 promotes filopodia formation. (C) and (D) For both cell lines, (C) the number and (D) length of filopodia were determined. (E) G/F-actin ratios from HAP1 control and NAA80 KO1 cells. Asterisks represent significance determined by Student' s t-test: * p≤0.05; ** p≤0.01; *** p ≤0.001. Shown are the means±SD. (F) and (G) Confocal images (top) and STED zoom-in frames of lamellipodia (bottom). The number of cells containing at least one clearly distinguishable lamellipodium supported by filopodia/microspikes was determined. Since HAP1 cells typically grow in clusters, only cells at the borders of clusters (i.e. with a front facing away from the cluster) were considered, as indicated by the numbering. Lamellipodia-positive cells are indicated by green numbers. (H) Quantification of lamellipodia-positive cells.

FIG. 9: Actin Nt-acetylation affects filament elongation and depolymerization. (A) Coomassie and immunoblot analysis of cytoplasmic actin purified from wild type and NAA80 KO cells. 100% Nt-acetylated a-skeletal actin (15) is shown for reference. (B) The polymerization rate of actin alone (2μM actin (6% pyrene-labelled)) is unchanged±Nt-acetylation. Data shown as the average curve from the number of independent experiments indicated in each panel, with standard deviation error bars in lighter color (Ac-actin, blue; non-Ac-actin, red). (C) Elongation actin alone (1.5 μM filament seeds; 0.5 μM actin (6% pyrene-labelled)). The barbed end elongation rate of actin filament seeds is ˜2.2-fold faster for Ac-actin than non-Ac-actin. The polymerization of Ac-actin and non-Ac-actin in the absence of seeds are shown as controls. (D) Depolymerisation actin alone (0.1 μM filament seeds; (6% pyrene-labelled). The depolymerization rate of actin filament seeds is ˜1.7-fold faster for Ac-actin than non-Ac-actin. (E) Nucleation and branching (Arp⅔ complex). 2 μM actin (6% pyrene-labelled). 100 nM N-WASP WCA [Arp⅔ complex]. The concentration dependence of polymerization rates of actin assembly by Arp⅔ complex (nucleation and branching) shows no difference for Ac-actin vs. non-Ac-actin. Shown is the mean±s.e.m of three independent experiments. (F) Nucleation and barbed end capping (mDia2). 2 μM actin (6% pyrene-labelled). [mDia2]. The concentration dependence of polymerization rates of actin assembly induced by mDia2 (nucleation and barbed end capping) is unchanged±Nt-acetylation. (G) Nucleation and elongation (mDia1). 2 μM actin (6% pyrene-labelled). [mDia1]. The concentration dependence of polymerization rates of actin assembly induced by mDia1 (nucleation and barbed end elongation) is>2-fold faster for Ac-actin than non-Ac-actin. (H) Formin elongation from profilin1-actin. 1.5 μM filament seeds. 20 nM mDia1, 0.5 μM actin (6% pyrene-labelled), 1.5 μM profilin1. The elongation rate of filament seeds by mDia1 from profilin1-actin is ˜35% faster for Ac-actin than non-Ac-actin. For both actins, the elongation rate of mDia1 from profilin1-actin is faster relative to the elongation rate of actin seeds alone (shown as controls). (I) Formin elongation from profilin2-actin. 1.5 μM filament seeds. 20 nM mDia1, 0.5 μM actin (6% pyrene-labelled), 1.5 μM profilin2. In contrast, barbed end elongation by mDia1 from profilin2-actin is unchanged for non-Ac-actin and reduced ˜40% for Ac-actin relative to the elongation rate of actin seeds alone.

FIG. 10: (A) IC50 values for inhibitors measured in an Nt-acetylation inhibitor assay for Naa80. (B) Ki-values for CoA-Ac-DDDI-NH₂ (SEQ ID NO: 6) for different human NATs measured in an Nt-acetylation inhibitor assay. All reactions were performed at least three times and in triplicate; errors bars represent standard deviation of each measurement.

### EXAMPLES

The present invention is further illustrated by the following Examples, in which parts and percentages are by weight and degrees are Celsius, unless otherwise stated. It should be understood that these Examples, while indicating preferred embodiments of the invention, are given by way of illustration only. From the above discussion and these

Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

### Example 1: Synthesis of Bisubstrate Analogues

Bisubstrate analogues were prepared by Fmoc-based solid phase peptide synthesis (SPPS) on a Biotage® Initiator+ Alstra™ (Biotage, Sweden) automated microwave peptide synthesizer using a ChemMatrix Rink amide resin (0.47 mmol/g loading) on a 0.2 mmol scale. Each amino acid (4 equiv.) was coupled using DIC (4 equiv.) and oxyma (4 equiv.) or HCTU (4 equiv.) and DIPEA (8 equiv.) in dimethylformamide (DMF) with microwave heating at 75° C. for 5 min. Removal of the Fmoc protecting group was facilitated by treating the resin with piperidine (20% in DMF) at room temperature for 3+10 min. Following Fmoc-deprotection of the N-terminal amino acid, resin-bound peptides were treated with bromoacetic acid (8 equiv.) and DIC (8 equiv.) in DMF for 1 hr as previously described. Final deprotection and cleavage from the solid support was facilitated by treatment of the resin with a mixture of trifluoroacetic acid (TFA), triisopropylsilane (TIS) and water (95:2.5:2.5 v/v, 12.5 mL/g of initial resin used) for 2 h. For the Met-containing inhibitor, a mixture of TFA, TIS, ethane dithiol and water (92.5:2.5:2.5 v/v, 12.5 mL/g of initial resin used) was used. The resin was removed by filtration and washed with an additional portion of cleavage cocktail (12.5 mL/g of initial resin used). The combined TFA fractions were concentrated until approximately 5 mL of the solution remained upon which diethyl ether was added to facilitate precipitation. The diethyl ether was carefully removed using a pipette, and the residues were washed with two portions of fresh diethyl ether. The crude bromoacetyl peptides were dried under vacuum, purified by semipreparative reverse-phase high performance liquid chromatography (RP-HPLC) and lyophilized.

Purified bromoacetyl peptides (typically 10 mg) and CoA trilithium salt (2 equiv.) were dissolved in triethylammonium bicarbonate buffer (1 M, pH 8.5) and left at room temperature for 16 hr. Finally, the CoA-Ac-peptides were purified by semi-preparative RP-HPLC and lyophilized to give colorless powders. All bisubstrate analogues were purified to a purity of>95% (UV 220 nm) and their structures were confirmed by ESI-MS and NMR analyses.

### Example 2: Inhibition of NatA using Bisubstrate Analogues

A number of bisubstrate analogues were prepared as described in Example 1 with potential inhibitory properties against NatA.

The IC50 values for these compounds were tested as described in Foyn H, et al. (2013) “Design, Synthesis, and Kinetic Characterization of Protein N-Terminal Acetyltransferase Inhibitors”. ACS Chem Biol 8:1121-1127; and Foyn H, et al. (2017) “DTNB-Based quantification of in vitro enzymatic N-terminal acetyltransferase activity”, Methods Mol. Biol. 1574:9-15. Briefly, a colorimetric acetylation assay, 5,5′-dithiobis-(2-nitrobenzoic acid) (DTNB) assay was used to measure the catalytic activity of NATs after incubation with NAT inhibitors. In the DTNB assay, thiols present in the enzymatic product, CoA, cleaves DTNB and produces 2-nitro-5-thiobenzoate (NTB-), which absorbs light with a wavelength of 412 nm. NTB- forms in a 1:1 molar ratio to the thiol groups present in the sample. Therefore, NTB- was quantified by measuring the absorbance at 412 nm and indirectly measures peptide Nt-acetylation. Recombinant purified NAT enzyme was mixed with different substrate peptides (300 uM, custom-made) and Ac-CoA (300 uM) in acetylation buffer (50 mM Tris/HEPES pH 8.5-7.4, 100 mM NaCl, 10% glycerol and 0.2 mM EDTA) and with inhibitor concentration (0-500 uM). After 30 min-60 min incubation at 37° C., the enzyme activity was quenched with quenching buffer (3.2 M guanidinium-HCl, 100 mM sodium phosphate dibasic pH 6.8). Triplicates were run for all positive samples, while negative controls were duplicated. As a negative control, reactions without enzyme were incubated at 37° C. before the reaction was quenched and added enzyme. In order to measure CoA production, 2 mM DTNB (dissolved in 100 mM sodium phosphate dibasic pH 6.8 and 10 mM EDTA) was added to the quenched reaction and the absorbance at 412 nm was measured with a spectrophotometer (Epoc). Background absorbance (determined in negative controls) was subtracted from the absorbance determined in each individual reaction. Thiophenolate production was quantified assuming ε=13.7×103 M−1 cm−1. For NatA and NatB enzyme complex inhibition, immunoprecipitation was performed using Naa15 ab (custom made, Arnesen T, Anderson D, Baldersheim C, Lanotte M, Varhaug J E, Lillehaug J R. (2005) Identification and characterization of the human ARD1-NATH protein acetyltransferase complex. Biochemical J 386(Pt 3):433-43.) or Naa20 ab (Nat5, abnova) respectively and protein A/G magnetic agarose beads (Millipore). A similar Nt-acetylation reaction was performed as previously described and the enzymatic activity was quenched with a final concentration of 1% TFA and analyzed by HPLC (Evjenth R, Van Damme P, Gevaert K, Arnesen T (2013) HPLC-based Quantification of in vitro N-terminal Acetylation. Methods Mol Biol 981:95-102.).

The results are shown in the table below.

TABLE 1
Overview of IC50 values of all inhibitors tested against NatA

| Compound | IC50 (μM) | SEQ ID NO: |
|---|---|---|
| CoA-Ac-SESS | 15.4 ± 0.97 | 26 |
| CoA-Ac-SASE | 62.4 ± 35.5 | 27 |
| CoA-Ac-AASE | 247 ± 10.5 | 28 |
| CoA-Ac-SSSE | 9.47 ± 0.21 | 29 |
| CoA-Ac-SAAE | 12.1 ± 1.12 | 30 |
| CoA-Ac-SSAE | 4.7 ± 0.28 | 31 |
| CoA-Ac-SYAE | 36.2 ± 0.85 | 32 |
| CoA-Ac-SSAA | 5.3 ± 0.24 | 33 |
| CoA-Ac-SSME | 1.28 ± 0.18 | 34 |
| CoA-Ac-SSMP | 0.82 ± 0.11 | 3 |
| CoA-Ac-SSMPV | 2.1 ± 0.42 | 35 |
| CoA-Ac-SS(Nle)P | 1.46 ± 0.29 | 4 |
| CoA-Ac-PS(Nle)P | >>1000 | 36 |

All of the above compounds comprised C-terminal amide groups.

Although CoA-Ac-SSMP (SEQ ID NO: 3) was found to be a very potent inhibitor of NatA, there were problems synthesizing it; this was due in part to a side-reaction of the methionine side chain. A norleucine (Nle) residue was therefore tried in place of methionine in order to try to circumvent the synthesis problem. CoA-Ac-SSNleP (SEQ ID NO: 4) showed only a small increase in IC50 value compared to CoA-Ac-SSMP (SEQ ID NO: 3), but it was much more easily synthesized. The CoA-ac-SSNleP inhibitor (SEQ ID NO: 4) also gave the same phenotype in zebrafish as SSMP (SEQ ID NO: 3).

Both CoA-Ac-SSNlePK-folic acid (SEQ ID NO: 23) and CoA-Ac-SSNlePK-CPP9 (SEQ ID NOs: 23 and 25) gave comparable phenotypes as CoA-Ac-SSNleP-NH2 (SEQ ID NO: 4) when injected into zebrafish embryos.

### Example 3: Selectivity of Bisubstrate Analogues for NatA

In order to ensure selectivity towards NatA, the CoA-Ac-SSNIeP (SEQ ID NO: 4) was tested against other NATs. Using the Cheng-Prusoff equation for competitive inhibitors, the dissociation constant, Kᵢ, was calculated from IC50 values and [S]/Kₘ ratios for all the NATs.

The results (see Table 2 below) clearly indicate that CoA-Ac-SSNleP (SEQ ID NO: 4) selectively inhibits NatA because it is at least 100-fold more potent towards NatA than all the other NATs tested.

TABLE 2
Kᵢ values for CoA-Ac-SSNleP (SEQ ID NO: 4) against all NATs

| Compound | Kᵢ (μM) |
|---|---|
| NatA | 0.143 ± 0.03 |
| Naa10 | 98.9 ± 443 |
| NatB | 41.6 ± 51.2 |
| Naa30 | 132.1 |
| Naa40 | 41.9 ± 6.4 |
| Naa50 | 17.7 ± 7.4 |
| Naa60 | 3.1 ± 0.8 |

### Example 4: Use of NatA Bisubstrate Analogues in Cell-Proliferation Assay

Cancer cell culture screening of the cell permeable NatA bisubstrate-analogue inhibitors (NatA inhibitor+negative control CoA-Ac-PSNleP (SEQ ID NO: 36)) is carried out. Studies on the NatA inhibitors include, in a step-wise manner, phenotypic determination of cancer cells (cell viability etc.), in depth characterization of activated signalling pathways as well as analysis of N-terminal acetylation status by COFRADIC analyses. Several different cancer cell types are assessed for their sensitivity to NatA-inhibitors, including thyroid cancer cells (Gromyko D, et al. (2010) “Depletion of the human N-terminal acetyltransferase A (hNatA) induces p53-dependent apoptosis and p53-independent growth inhibition”. Int. J. Cancer 127(12): 2777-89) breast cancer cells, lung cancer cells, and prostate cancer cells. The NCI-60 cancer cell panel in collaboration with SINTEF (Trondheim, Norway) is considered to get a broad cancer profile. Cells are plated in 96 or 384 well plates and grown for 16 hours prior to the addition of test compounds so as to allow cells to adhere to the well and begin growing. A 10-point concentration series, spanning at least 3 log units of concentration, of our NatA inhibitor set is added to cells in assay plates at a constant DMSO concentration empirically determined to be below the maximum tolerated dose. Cells are incubated with inhibitors for at least 2-3 population doublings before measuring the viability response. The widely accepted Z′-factor and minimum significant ratio (MSR) metrics are used to quantitatively assess our assays' reproducibility and sensitivity. The activity of our NatA inhibitors in H1299 (lung cancer), CAL-62 and 8305C cells (anaplastic thyroid carcinomas) are initially tested because they have been shown to be sensitive to NatA dosage (Lim J H, Park J W, Chun Y S (2006) Cancer Res 66(22):10677-82.; Gromyko D, Arnesen T, Ryningen A, Varhaug J E, Lillehaug J R (2010) Int J Cancer 127(12):2777-89.). To demonstrate selectivity, each compound's activity to Nthy-ori 3.1 cells (SV40 immortalized thyroid follicular epithelial cells) is compared. The selectivity of each compound is defined as the ratio of the IC50 in CAL-62/8350C to the IC50 in Nthy-ori3.1. The EC50 is defined as the compound concentration that results in 50% inhibition of cell proliferation/viability relative to cells treated with control agents (e.g. negative: DMSO, positive: cytotoxic agent).

Our hypothesis is that the newly-discovered NatA inhibitors will possess a selective (>10-fold), concentration dependent activity in the CAL-62 and 8305C cells relative to the Nthy-ori3.1 cells. Combination therapies that enhance efficacy or permit reduced dosages to be administered have seen great success in a variety of therapeutic applications. Published data indicates that NatA dosage regulates the sensitivity of thyroid carcinoma cells to cytotoxic agents, including Daunorubicin and KillerTRAIL (Gromyko D, Arnesen T, Ryningen A, Varhaug JE, Lillehaug JR (2010) Int J Cancer 127(12):2777-89.). This observation supports a hypothesis that NatA inhibitors would be useful in combination with clinically used compounds. Synergistic combinations are of particular interest, as they can i) enhance the activity of the mixture relative to that expected from additivity; ii) increase potency; or iii) enhance efficacy. For these synergy experiments, a traditional 8-point matrixed dose—response analysis is used that tests pairwise combinations of NatA inhibitors with classic cytotoxic agents (e.g. daunorubicin) or molecular targeted therapies (KillerTRAIL, troglitazone, bortezomid). Synergy will be assessed by Bliss and Loewe models as described (Severyn B, Liehr R A, Wolicki A, Nguyen K H, Hudak E M, Ferrer Met al. (2011) ACS Chem Biol 6:1391-1398.). Synergistic combinations that enhance potency and efficacy are prioritized for further characterization.

### Example 5: Plasmid Construction, Recombinant Protein Expression, and Purification of hNAA80

hNAA80/hNAT6 (Gene ID: 24142) was cloned from human HEK293 cDNA by use of Transcriptor Reverse Transcriptase (Roche) using the following primers: NAA80 sense primer (5′-CAACATGCAAGAGCTGACTC-3′ SEQ ID NO: 37) and NAA80 antisense primer (5′-GATGTCTTTTTCCATCCAGAATATG-3′ SEQ ID NO: 38). The PCR product containing the CDS was inserted into the TOPO TA vector pcDNA 3.1/V5-His TOPO (Invitrogen) using the provided kit and resulting in the plasmid pNAA80-V5. Plasmid hNAA80-eGFP was constructed using pNAA80-V5 as template and restriction sites Nhel and Kpnl in peGFP-N1. The MBP-HishNAA80 fusion protein (MBP-Naa80) was constructed by subcloning hNAA80 from the plasmid pNAA80-V5 into the pETM-41 vector using the restriction enzymes Ncol and Acc65I resulting in pETM-41-hNAA80. The plasmid was transformed into E. coli BL21 Star™ (DE3) cells (Invitrogen) by heat shock. A 300 mL cell culture was cultivated in Luria-Bertani (LB) medium at 37° C. until an OD600 of 0.6 was reached and subsequently transferred to 20° C. Protein expression was induced by addition of 1 mM isopropyl-β-D-thiogalactopyranoside (IPTG). After 16 h, the cells were harvested by centrifugation and the pellets were stored at −20° C. For purification, the E. coli pellets were thawed at 4° C. and the bacterial cells lysed using mechanical disruption by a French Press (1000 psi pressure) in lysis buffer (50 mM Tris-HCl (pH 8.0), 300 mM NaCl, 20 mM imidazole, 1 mM DTT, 1 x EDTA-free protease inhibitor cocktail (Roche)). After centrifugation (40,000 x g, 25 min, 4° C.), the cell extract was applied on a metal affinity FPLC column (HisTrap HP, GE Healthcare). MBP-Naa80 was eluted with 300 mM imidazole in 50 mM Tris (pH 8.0), 300 mM NaCl, 1 mM DTT. Recombinant protein containing fractions were pooled and further purified via size exclusion chromatography (Superdex 200, GE Healthcare) and purity was determined by analysis of Coomassie stained SDS-PAGE gels. The protein concentrations were determined by absorption at 280 nm using a NanoDrop1000 spectrophotometer (Peqlab, Germany).

### Example 6: [¹⁴C]-Ac-CoA based N-Terminal Acetyltransferase Assay using Synthetic Oligopeptides

Whilst Nat6/Naa80 has previously been described as a putative NAT (based on sequence analysis that suggested the presence of a NAT-specific GNAT-fold), no specific substrate has previously been described.

Here we studied the Nt-acetylation activity of Naa80 towards a representative selection of peptides, using a [¹⁴C]-Ac-CoA based N-terminal acetyltransferase assay using synthetic oligopeptides.

The assay was performed as described previously (Drazic A & Arnesen T (2017) [14C]-Acetyl-Coenzyme A-Based In Vitro N-Terminal Acetylation Assay. Methods Mol. Biol. 1574:1-8). Briefly, the immuno-precipitates were mixed with 200 μM selected synthetic oligopeptides (Biogenes, Germany), 50 μM isotope-labelled [¹⁴C]-Ac-CoA (Perkin Elmer) in a total volume of 25 μL acetylation buffer (50 mM Tris (pH 7.4), 1 mM EDTA, 1 mM DTT, 10% (v/v) glycerol) The samples were incubated at 37° C. and shaking at 1000 rpm in a Thermomixer block for 60 min. The magnetic beads were removed and the supernatants transferred onto P81 phosphocellulose filter disks (Millipore). The filters disks were washed three times with washing buffer (10 mM HEPES (pH 7.4)) and dried before they were added to 5 mL scintillation cocktail Ultima Gold F (Perkin Elmer). The incorporated [¹⁴C]-Ac was determined by a Perkin Elmer Tri-Carb 2900TR Liquid Scintillation Analyzer.

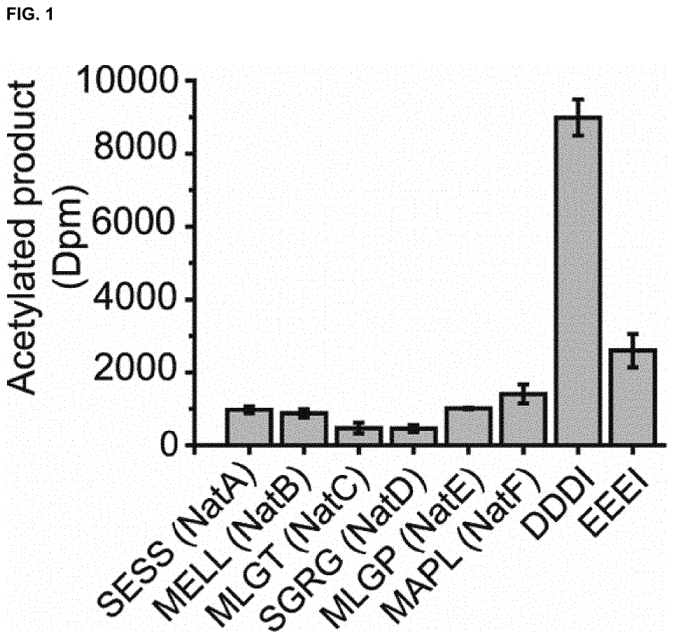

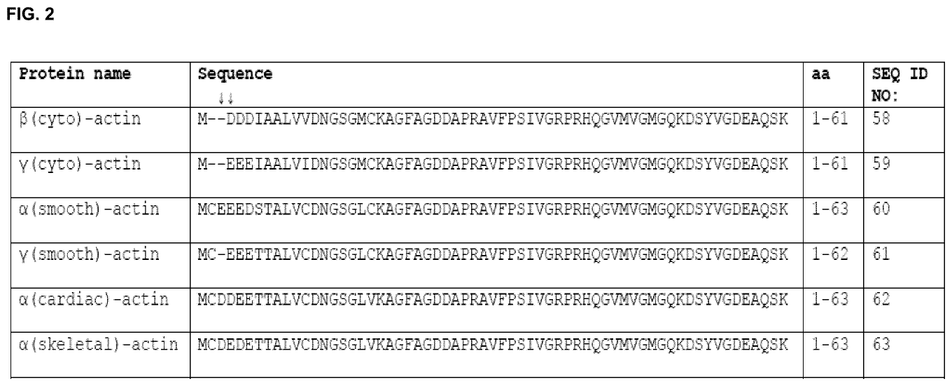

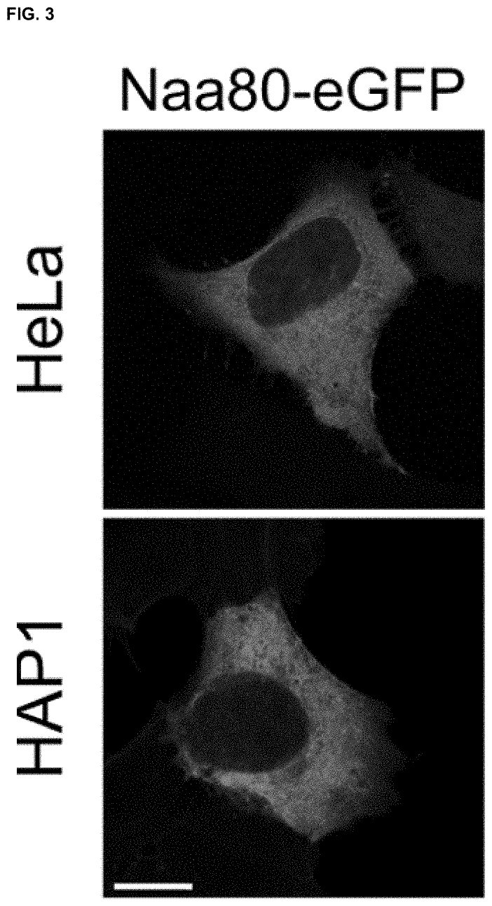

The results are shown in FIG. 1. The results revealed a marked preference of Naa80 for peptides with acidic N-termini (e.g. DDDI (SEQ ID NO: 6) and EEEI (SEQ ID NO: 7)). Among all the proteins expressed in animals, these N-termini uniquely correspond to those of cytoplasmic β- and γ-actin, after the intermediate step of post-translational cleavage of the N-terminal acetyl-methionine. This is illustrated in FIG. 2 which shows a sequence alignment (first 61-63 amino acids) of the six human mature actin isoforms.

### Example 7: Expression of Naa80-eGFP in Human HAP1 Cells

The expression pattern of Naa80 in human cells was evaluated as follows:

HAP1 cells were obtained from Horizon Genomics and cultured as recommended. NAA80 knockout and wildtype HAP1 cells (Horizon C631) were grown in Iscove's Modified Dulbecco's Medium with the addition of 10% fetal bovine serum and 1 penicillin/streptomycin. Prior to use in experiments, all HAP1 cell lines were passaged until diploid status was confirmed by an Accuri BD C6 flow cytometer using propidium iodide staining. HeLa cells (ATTC CCL-2) were cultured in DMEM/10% FBS at 37° C. and 5% CO₂. Cells were seeded on plates or coverslips and transfected with XtremeGene 9 as recommended. For localization and phenotype rescue studies, cells were imaged approx. 24 h and 48 h post- transfection, respectively.

The results are shown in FIG. 3. Naa80-eGFP expressed in human HAP1 cells displayed a diffuse cytosolic distribution and contrary to most NATs, it did not associate with ribosomes suggesting that it targets its substrate(s) post-translationally.

### Example 8: Identification of Naa80-Specific Substrates

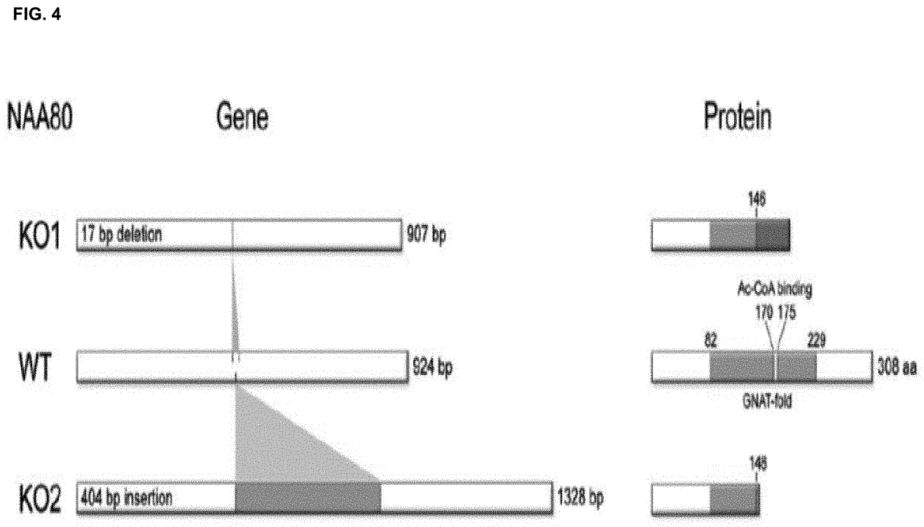

To identify Naa80-specific substrates in cells, we generated two HAP1 NAA80 knockout cell lines (KO1 and KO2) using the CRISPR/Cas9 system (FIG. 4).

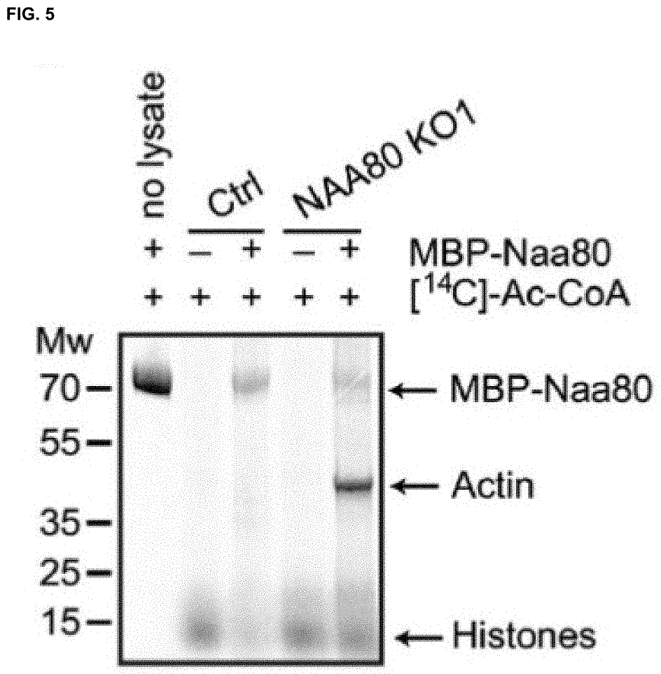

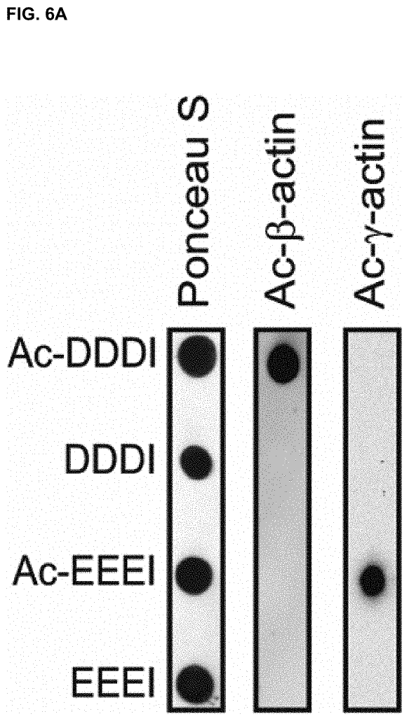

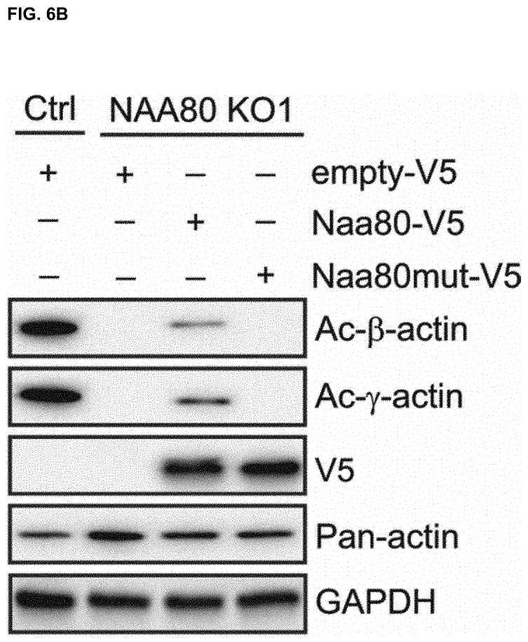

Phospho-imaging of NAA80 KO1 cell lysates treated with purified Naa80 and isotope-labeled [¹⁴C]-acetyl CoA revealed a single band at ˜43 kDa (FIG. 5), corresponding to the expected molecular mass of actin. Actin's identity was confirmed using N-terminal proteomics, by quantitatively assessing the Nt-acetylation status of 402 unique N-termini from control and NAA80 KO1 cell lysates. Only peptides corresponding to the fully processed N-termini of β- and γ-actin showed altered Nt-acetylation levels in knockout cells. Furthermore, both actin isoforms were 100% and 0% Nt-acetylated in control and NAA80 KO1 cells, respectively. Isoform- and Nt-acetylation-specific actin antibodies recognized a ˜43 kDa band in control but not NAA80 KO1 cells (FIG. 6A, 6B). The anti Ac-β-actin and Ac-γ-actin antibody signals reappeared in knockout cell lysates transfected with Naa80 (FIG. 6B), but not in cells transfected with a catalytically inactive mutant, Naa80mut (W105F, R170Q, G173D, Y205F) (FIG. 6B), confirming that a catalytically active Naa80 is absolutely required for actin Nt-acetylation in cells.

These results were corroborated for both knockout cell lines at the single cell level by immunofluorescence analysis, showing that only control cells and Naa80- eGFP-transfected knockout cells were positive for anti Ac-β-actin and Ac-γ-actin antibody staining.

### Example 9: Actin Nt-Acetylation Controls Cell Motility

Next, we explored the impact of actin Nt-acetylation in cells. Since the actin cytoskeleton is a major factor in cell motility, we tested HAP1 control and NAA80 knock-out cells in several independent migration assays. We found that the lack of actin Nt-acetylation resulted in increased motility of HAP1 NAA80 knockout cells.

Wound healing/gap closure and chemotaxis assays

For wound healing assays, cells were seeded in wells of silicone culture inserts (ibidi 80209) placed in slide wells (ibidi 80426). 90 μl of 300,000 cells/ml were seeded in each insert well, resulting in 100% confluency after approx. 24 hours at which insets were removed (creating a 400-450 μm wide gap), wells washed twice in medium and timelapse started using a Nikon TE2000 microscope with 10x objective. ImagePro plus was used for image processing to measure gap size in μm². Cell front velocity was calculated according to ibidi Application note 30. The total area of image was multiplied with the centerpiece approximation (the increase of the cell-covered area (in %) per time unit; this number was divided by the length of the image in μm and the resulting number divided by 2 for the two cell fronts), resulting in the normalized cell front velocity in μm/h. For scratch wounds using IncuCyte ZOOM imaging system, 200 μl of 40,000 cells/ml were seeded in 96-well Image lock plates (Essen BioScience) and scratch wounds were prepared with the accompanying scratch wound maker according to manufacturer protocols (creating a 700-μm wide gap). Wells were washed twice and filled with 300 μl medium. Wounds were imaged in the IncuCyte by 24 hours repeat scanning every 2 h in which 2 positions were imaged per well. Image analysis was performed in the IncuCyte ZOOM software using area masks for wound and cell covered area and calculations of average gap size. Cell front velocity was calculated as above. For chemotaxis assay in the IncuCyte ZOOM, cells were seeded in membrane-containing inserts of ClearView 96-well Cell migration plates with pored membranes. Cells were seeded at a density of 27,000 cells/ml, 60 μl/insert well in medium containing 1% FBS. After 3 h, media of different FBS concentrations were added to appropriate top or bottom wells to obtain desired final concentrations of FBS while keeping volumes equal. The membranes were imaged at top and bottom using 10x objective and 24 h repeat scanning every 2 h. Analysis was performed in the IncuCyte ZOOM software measuring cell covered area at top and bottom. Numbers were expressed as amount of cells migrated per tin percent of initial top value. Data were background-subtracted and corrected for growth differences.

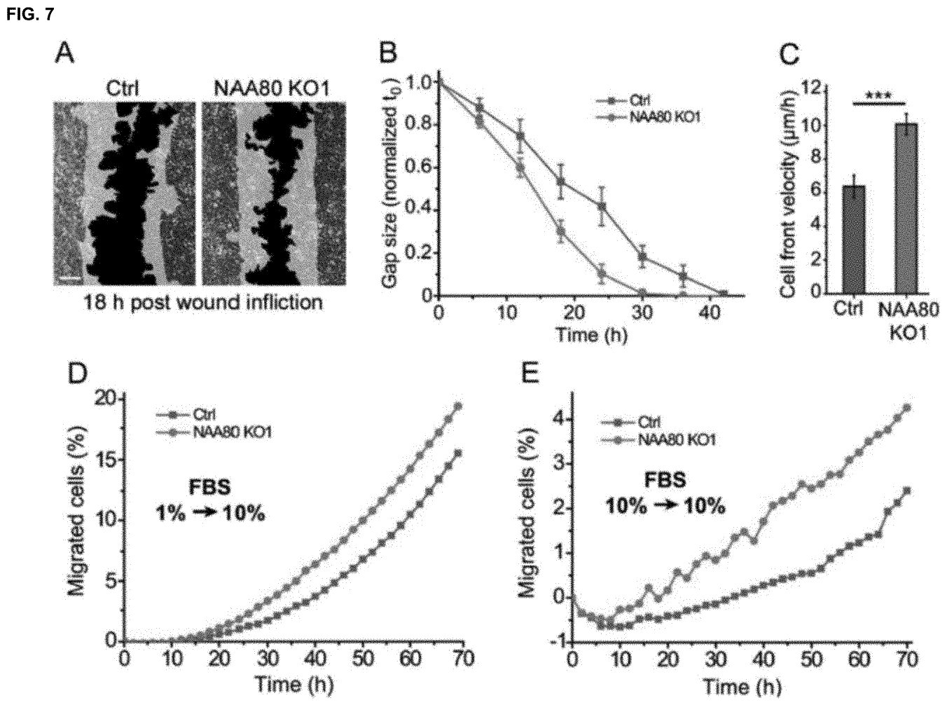

In the wound-healing assays, NAA80 KO1 cells closed the gap approximately 12 h faster than control cells (FIG. 7A, 7B), with cell-front velocity increasing from 6 to 10 μm/h (FIG. 7C). We observed similar results for both knockout cell lines in an IncuCyte ZOOM system for live-cell imaging, showing an increase of cell-front velocity from 7 to 9 μm/h and 11 μm/h for NAA80 KO1 and KO2 cells, respectively. To investigate the migratory properties of single cells, we performed a chemotaxis assay by seeding cells on a transparent pored membrane in medium containing 1% FBS. Cells were attracted to the bottom side by a medium containing 10% FBS, and the amount of migrated cells was normalized to the growth rates of each cell line. NAA80 KO1 cells showed approximately a 50% increase in chemotactic motility compared to control cells (FIG. 7D). In addition to the chemotactic assay for directed migration, we also measured random cell migration in parallel wells in which both chambers contained 10% FBS. In this assay, we also observed increased random motility of NAA80 KO1 cells (FIG. 7E), consistent with an increased capacity of these cells to migrate. Similar results were obtained with NAA80 KO2 cells.

Taken together, these data demonstrate that cells deficient in actin Nt-acetylation have increased motility.

### Example 10: Actin Nt-Acetylation Affects Cytoskeletal Morphology

The actin cytoskeleton not only regulates cell motility, but also cell shape and morphology. Thus, we compared the morphology of the actin cytoskeleton in HAP1 control and NAA80 KO cells by phalloidin staining.

Phalloidin-based phenotype characterizations

Lamellipodia phenotype was identified in cells seeded at 70,000 cells/ml in 24-well plates, fixed approx. 24 h post seeding in 3% PFA in cytoskeleton buffer (10 mM MES pH 6.1, 150 mM NaCl, 5 mM EGTA, 5 mM glucose, 5 mM MgCl₂), washed in CB, permeabilized in 0.1% Triton for 10 min, and stained with Rhodamine phalloidin or Phalloidin-Atto-647N, for counting and confocal/STED respectively. Cells at the edge of clusters were counted as either positive or negative for the presence of lamellipodia (mitotic cells were not considered and neither were lamellipodia on top of neighboring cells). Experiment was repeated three times and at least 500 cells were counted per cell line per independent repetition. Filopodia phenotype analysis was performed by seeding cells at 25-50,000 cells/ml in 48-well plates, fixed approx. 24 h post seeding in 3% PFA in 0.1 M phosphate buffer for 30 min and subsequently permeabilized with 0.1% Triton for 10 min, and stained with Rhodamine phalloidin. Filopodia number was counted on isolated cells and length measurements done using the imageJ software (National Institute of Health, Bethesda, MD, USA).

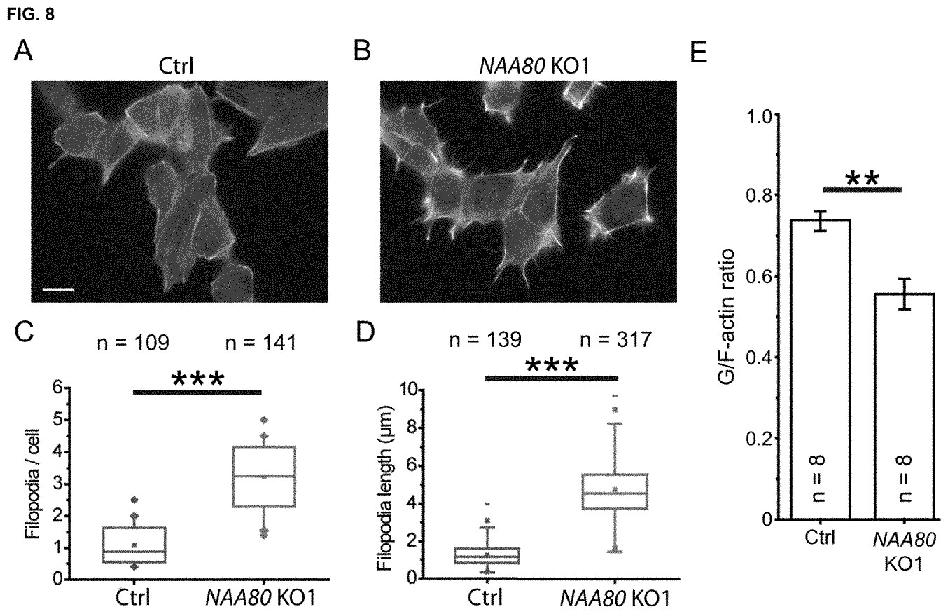

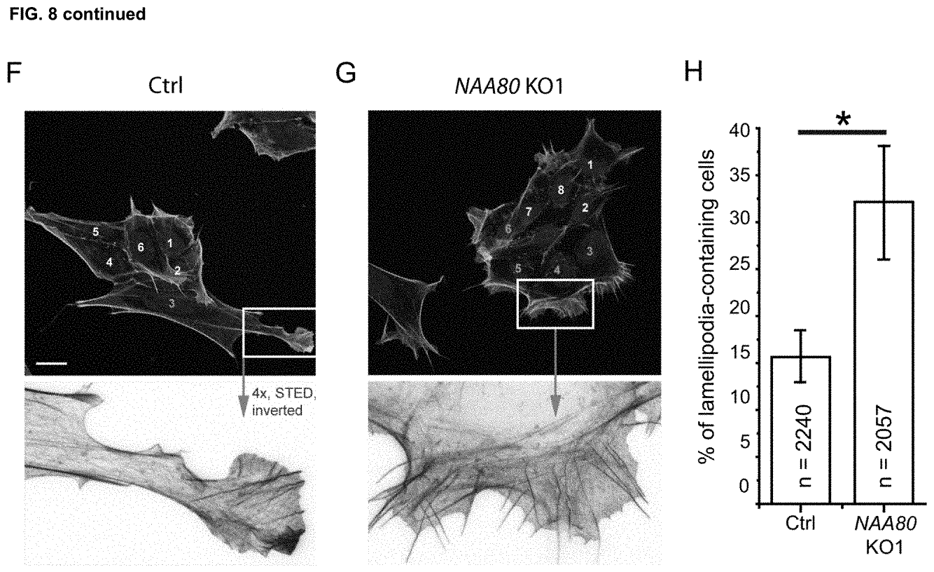

Based on the hypermotility phenotype of NAA80 KO cells, we were specifically interested in the formation of cell protrusions, such as lamellipodia and filopodia that are usually linked to cell motility. As anticipated, NAA80 KO cells displayed an increase in both the number and length of filopodia-like structures (FIG. 8A-D). These cells showed an approximate 4-fold increase in the number of filopodia-like structures, defined as finger-like protrusions with a length ≥0.5 μm (FIG. 8C). The average filopodia length increased from 1 pm in control cells to 5 μm in NAA80 KO cells (FIG. 8D). Confirming the specificity of this phenotype, the reexpression of Naa80 in NAA80 KO cells reduced the number of filopodia-like structures to near control levels, whereas the expression of the catalytically inactive mutant Naa80mut did not. As with other experiments described above, the NAA80 KO2 clone showed a similar behaviour. Consistent with the apparent elevated number of filamentous actin structures in NAA80 KO cells, we observed a significant reduction in the ratio of globular to filamentous (G/F)-actin (21) from 0.73 in HAP1 control cells to 0.55 in NAA80 KO1 cells (FIG. 8E). This effect was reversed by the expression of wild-type Naa80 but not Naa80mut in both NAA80 KO cell lines, demonstrating that Naa80-dependent actin Nt-acetylation directly impacts the transition between G- and F-actin. Since we observed a hyper-motility phenotype for NAA80 KO cells (FIG. 8), we quantified the number of cells showing at least one distinguishable lamellipodium supported by microspikes or filopodia-like structures. In order to efficiently study these cell protrusions in HAP1 cells, which typically grow in clusters, we included only cells at the borders of the clusters in quantifications. In apparent agreement with the increased cell front velocity in the motility assays, we observed that NAA80 KO cells formed twice as many lamellipodia than control cells (32% vs. 16%) (FIG. 8F-H). These results further emphasize the importance of actin Nt-acetylation in the control of cytoskeletal dynamics.

### Example 11: Nt-Acetylation-Dependent Actin Polymerization and Stability

To address the mechanism behind the altered cytoskeleton organization phenotypes of NAA80 KO cells, we analyzed the recovery rates of cytoskeletal structures in cells treated with the actin depolymerizing drug Latrunculin A (LatA).

Complete depolymerization of filamentous actin (F-actin) was achieved by incubating cells with 500 nM Latrunculin A (LatA) for 1 h at 37° C. Cells were either fixed with PFA (control) or washed with culture medium to remove LatA and further incubated in drug-free medium at 37° C. Samples were taken every 2 min, fixed and permeabilized with 0.2% Triton X-100 (in PBS) for 10 min and F-actin probed with 500 nM phallodin Atto 647N (STED microscopy) for 30 min.

Within 60 min of LatA treatment, the actin appeared to be fully depolymerized in control and NAA80 KO cells, and washout of the drug resulted in the recovery of actin filament structures, but the time of recovery was significantly delayed for NAA80 KO cells compared to control cells. Specifically, the formation of actin filament structures with an average length of 1 μm was delayed about 4 mins compared to control cells, consistent with a direct role of actin Nt-acetylation in actin polymerization.

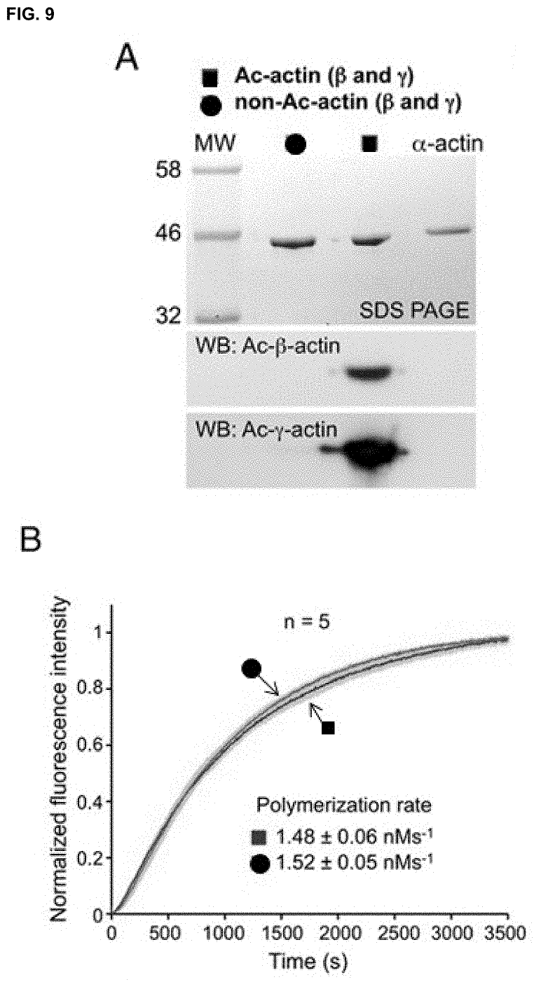

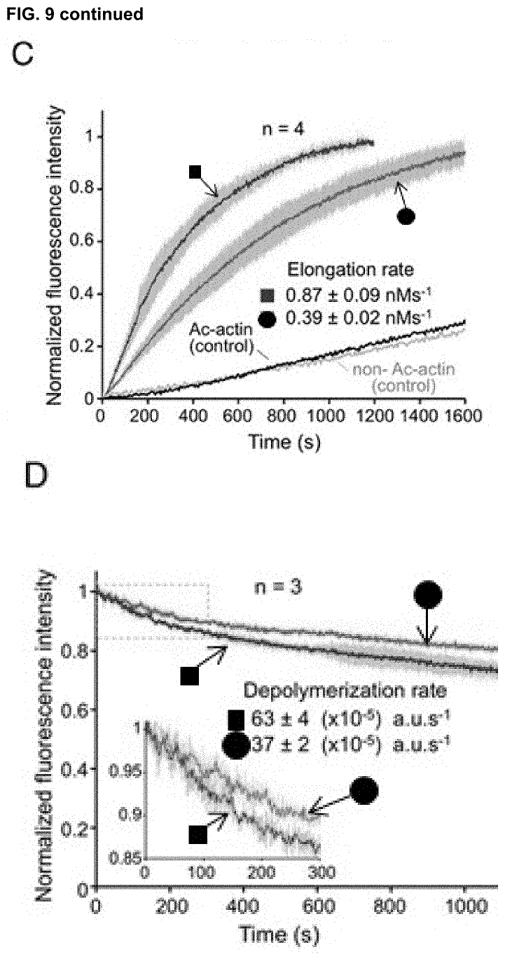

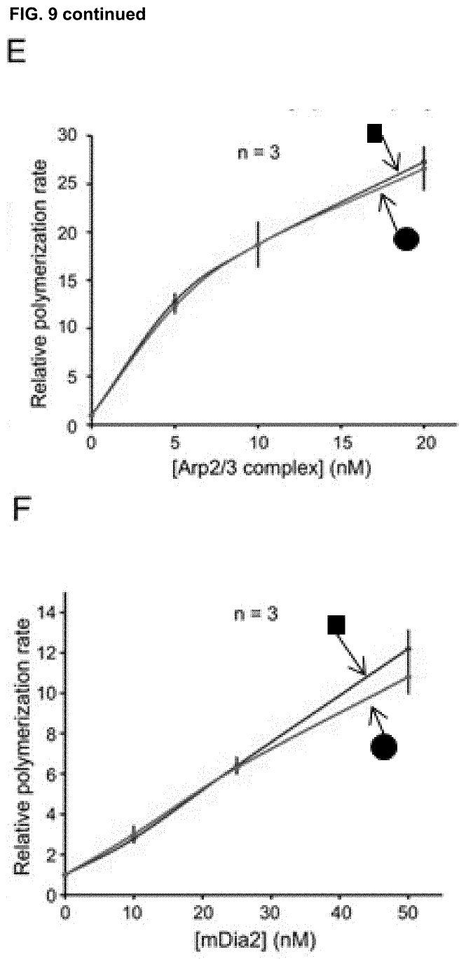

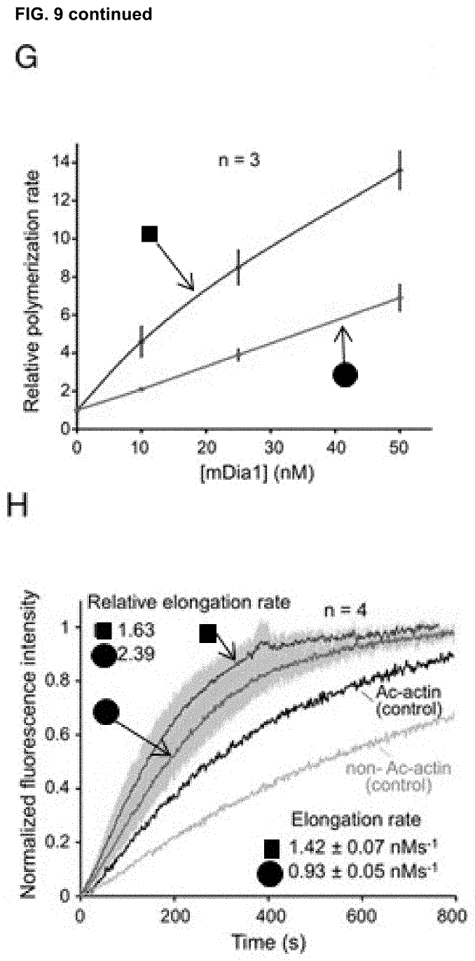

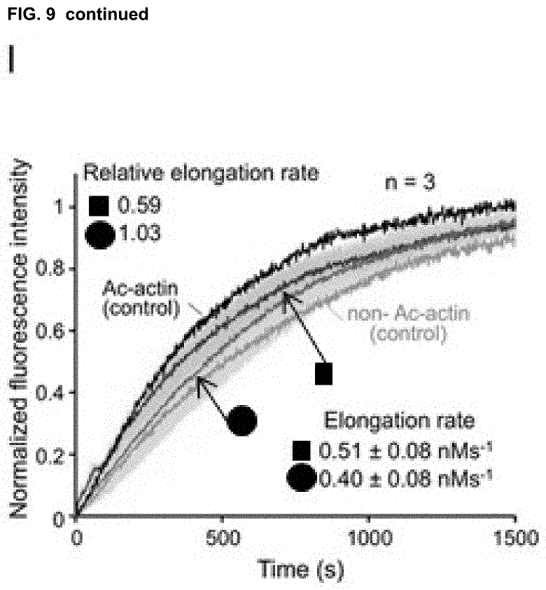

We next explored the in vitro effect of actin Nt-acetylation on the polymerization/depolymerization properties of actin alone or in the presence of some of the most common actin assembly factors in cells. Cytoplasmic actin (a mixture of β and γ isoforms) was purified from control and NAA80 KO cells (FIG. 9A). The time-course of actin polymerization measured using the pyrene-actin polymerization assay revealed no significant difference in the polymerization rate of Nt-acetylated or unacetylated actin (FIG. 9B). However, the elongation of 1.5 μM filament seeds in the presence of 0.5 μM actin monomers (i.e. above the critical concentration for monomer addition at the barbed end but below that of the pointed end) was more than 2-fold faster for Ac-Actin than non-Ac-Actin (FIG. 9C). In contrast, the depolymerization rate of 0.1 μM filament seeds (i.e. below the critical concentration of both the pointed and barbed ends) was ˜2-fold faster for Ac-actin than non-Ac-actin, suggesting that at least in the absence of other factors, actin filament structures formed by non-Ac-actin might be more stable (FIG. 9D).

Arp⅔ complex strongly enhances nucleation from the side of pre-existing filaments. We found no difference in the relative polymerization rate of Ac-actin and non-Ac-actin induced by Arp⅔ complex (FIG. 9E). We next analyzed two formins, mDia1 and mDia2, that while closely related in sequence have very different actin assembly properties. We observed no significant difference in the relative polymerization rates of Ac-actin and non-Ac-actin induced by mDia2 (FIG. 9D), a form in that has strong nucleation but poor elongation activity. In contrast, for mDia1, which has both strong nucleation and elongation activities, the polymerization rate was ˜2-fold higher for Ac-actin than non-Ac-actin (FIG. 9G). Because Nt-acetylation-dependent differences appear to be emphasized during form in-mediated elongation, we analyzed the role of profilin (isoforms 1 and 2), known to virtually stop pointed end elongation of actin alone while dramatically accelerating barbed end elongation by formins. As expected, mDia1 accelerated the elongation rate of filament seeds from profilin1-actin compared to the elongation of seeds from actin alone, but this effect was greater for Ac-actin than for non-Ac-actin (FIG. 9H). Curiously, no significant difference was observed with profilin2-actin (FIG. 9I), suggesting that mDia1 processes more efficiently profilin1-actin than profilin2-actin. We note, however, that these assays detect the incorporation into filaments of the small fraction of pyrene-labelled actin, which binds profilin with weaker affinity than unlabelled actin, such that the effects of profilin may be far more pronounced than indicated by the bulk assays.

Together, these results suggest that the differences in actin assembly between Ac-actin and non-Ac-actin emanate mainly from an Nt-acetylation-dependent increase in the rates of filament elongation and depolymerization, whereas nucleation appears to be mostly unaffected.

### Example 12: Naa80 Substrate Screening Assay

In order to investigate the in vitro activity of Naa80 further, an enzyme activity screening assay on purified Naa80 was undertaken using a broad substrate library (including amino acids, nucleosides, coenzymes, amines, saccharides, vitamins, antioxidants and peptides) modified from Kuhn et al. (Kuhn et al. (2013) “Broad-substrate screen as a tool to identify substrates for bacterial Gcn5-related N-acetyltransferases with unknown substrate specificity”. Protein Science 22(2):222-230). Among all of the potential substrates tested, only the peptides with N-terminal sequence of MDEL₂₄ (SEQ ID NO: 45), DDDI₂₄ (SEQ ID NO: 41) and EEEI₂₄ (SEQ ID NO: 40) were acetylated, as shown in the following table:

TABLE
Peptides tested as potential substrates for
Naa80 mediated acetylation

| Peptide | Product formation (μM) | SEQ ID NO: |
|---|---|---|
| EEEI₂₄ | 42.4 (±0.95) | 40 |
| MLGP₂₄ | 2.06 (±1.49) | 49 |
| MDEL₂₄ | 119 (±5.61) | 45 |
| MKKS₂₄ | 3.37 (±1.71) | 51 |
| DDDI₂₄ | 50.5 (±1.0) | 41 |
| SESS₂₄ | 1.14 (±3.35) | 43 |
| MTNK₂₄ | −4.25 (±1.75) | 48 |
| AVFA₂₄ | −0.96 (±0.84) | 44 |
| MAPL₂₄ | 2.28 (±0.61) | 50 |
| MELL₂₄ | 2.15 (±0.40) | 46 |

The 24-mer substrate peptides differed in their 7 N-terminal amino acids while the 17 C-terminal residues (RWGRPVGRRRRPVRVYP, SEQ ID NO: 39) were kept constant: EEEI₂₄ (EEEIAAL, SEQ ID NO: 40), DDDI₂₄ (DDDIAAL, SEQ ID NO: 41), MDDD₂₄ (MDDDIAA, SEQ ID NO: 42), SESS₂₄ (SESSSKS, SEQ ID NO: 43), AVFA₂₄ (AVFADLD, SEQ ID NO: 44), MDEL₂₄ (MDELFPL, SEQ ID NO: 45), MELL₂₄ (MELLSPP, SEQ ID NO: 46), MLGT₂₄ (MLGTGPA, SEQ ID NO: 47), MTNK₂₄ (MTNKSSL, SEQ ID NO: 48), MLGP₂₄ (MLGPEGG, SEQ ID NO: 49), MAPL₂₄ (MAPLDLD, SEQ ID NO: 50), MKKS₂₄ (MKKSYSG, SEQ ID NO: 51).

Substrate specificity studies in the linear range of enzyme concentration revealed that the best substrate for Naa80 was MDEL₂₄ (SEQ ID NO: 45), with a 5-fold higher product formation compared to the second best substrate, MDDD₂₄ (SEQ ID NO: 42), while its cellular substrate, processed β-actin, DDDI₂₄ (SEQ ID NO: 41) ranked third. Both MDEL (p65, SEQ ID NO: 8) and MDDD₂₄ (unprocessed β-actin, SEQ ID NO: 42) represent cellular NatB substrates.

### Example 13: Selective and Potent Inhibitors of Naa80

We then pursued Naa80 bisubstrate analogue inhibitors based on the results of the substrate screening and the sequences of the cellular substrates processed β- and γ-actin. We synthesized bisubstrate conjugates of Coenzyme A coupled to the tetrapeptides MDEL-NH₂ (SEQ ID NO: 8), DDDI-NH₂ (SEQ ID NO: 6), EEEI-NH₂ (SEQ

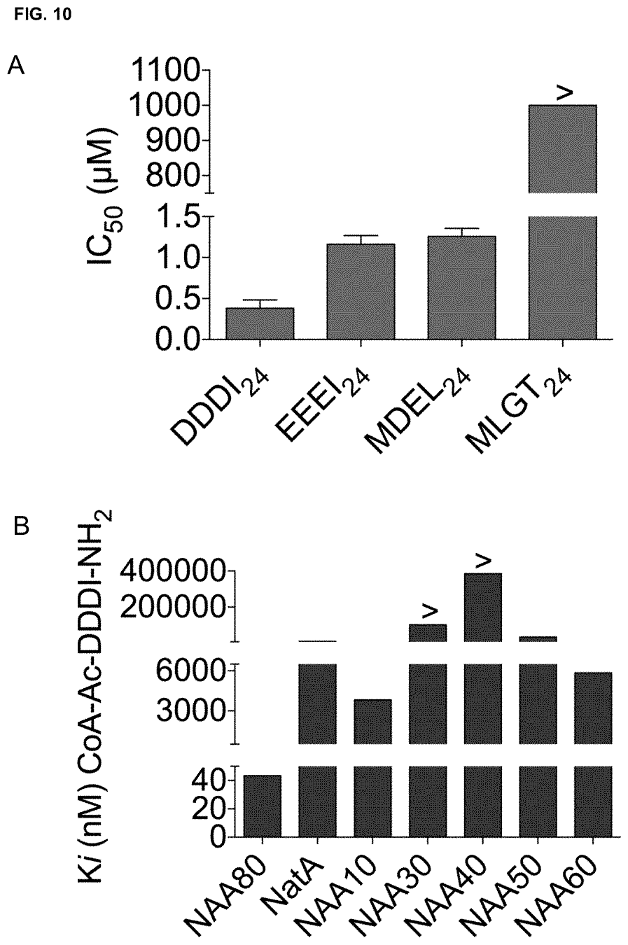

ID NO: 7), and MLGT-NH₂ (SEQ ID NO: 52) via an acetamide (Ac) linker. Inhibition studies revealed that CoA-Ac-DDDI-NH₂ was the most potent Naa80 inhibitor with an IC50 value of 0.38 μM, 3-fold and 3.3-fold more potent than CoA-Ac-EEEI-NH₂ (SEQ ID NO: 7) and CoA-Ac-MDEL-NH₂ (SEQ ID NO: 8), respectively (FIG. 10A). The negative control, CoA-Ac-MLGT-NH₂ (SEQ ID NO: 52), based on a NatC substrate, did not show detectable inhibition of Naa80 at the highest concentration tested of 1000 μM.

In order to determine the selectivity of the most potent Naa80 inhibitor, CoA-Ac-DDDI-NH₂ (SEQ ID NO: 6) was tested against a panel of human NATs (FIG. 10B). With a Kᵢ value of 43 nM, the inhibitor was established to be both potent and selective.

Interestingly, the monomeric Naa10, which has a known preference for acetylating acidic N-termini in vitro, was the second most inhibited NAT enzyme. However, CoA-Ac-DDDI-NH₂ (SEQ ID NO: 6),showed a 88-fold reduced potency for Naa10 compared to Naa80.

### Example 14: Further Inhibitors of Naa80

The following additional Naa80 bisubstrate analogue inhibitors were produced and tested as described above:

| SEQ ID NO: | Peptide | IC50 (μM) | StDev (μM) |
|---|---|---|---|
| 6 | DDDI | 0.38 | 0.10 |
| 7 | EEEI | 1.16 | 0.10 |
| 8 | MDEL | 1.26 | 0.09 |
| 53 | PDEL | 13.39 | 2.24 |
| 9 | DEDI | 3.12 | 0.33 |
| 10 | DEEL | 4.73 | 1.43 |
| 11 | EDDI | 0.12 | 0.05 |
| 12 | EDEI | 0.76 | 0.18 |
| 13 | EEDL | 1.76 | 0.52 |
| 14 | EEEL | 1.16 | 0.12 |
| 15 | DDEI | 2.67 | 0.67 |
| 16 | EDQL | 0.85 | 0.05 |
| 17 | ESEL | 11.50 | 3.23 |
| 18 | DEEI | 1.22 | 0.33 |
| 19 | EEDI | 0.17 | 0.04 |
| 20 | EDEL | 0.15 | 0.02 |
| 21 | QEEI | 2.23 | 0.47 |

1. A compound of formula Co-enzyme A-acetyl-SS(Nle)P-NH₂ (SEQ ID NO: 4).

2. A compound of Formula I:
[Co-enzyme A or analogue thereof]-Z1-Z2-Z3-Z4 (I)
wherein
Z1 is a linker moiety or is absent;
Z2 consists of a peptide or peptide-based moiety having the Formula II:
(SEQ ID NO: 1)
X1-X2-X3-X4 (II)
wherein
X1 and X2 are independently S or Hse;
X3 is M or Nle; a non-natural amino acid; a C₁₋₅ cyclic or non-cyclic alkyl group; or a C₁₋₅ ether group; and
X4 is P or is absent;
or Z2 consists of a peptide wherein the amino acid sequence of the peptide has the Formula III:
(SEQ ID NO: 2)
X5-X6-X7-X8 (III)
wherein
X5 is D, E, M or Q;
X6 is D, E or S;
X7 is D, E or Q; and
X8 is I or L;
Z3 is 0-30 amino acids; and
Z4 is a C-terminal group or is absent,
or a pharmaceutically-acceptable salt thereof.

3. The compound as claimed in claim 2, wherein the Co-enzyme A analogue is 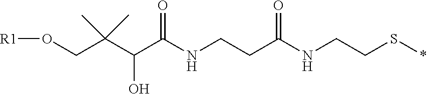
wherein R1 is hydrogen, a phosphate group, acetoacetate, alkyl, aralkyl or a cyclic alkyl.

4. The compound as claimed in claim 2, wherein Z1 is acetyl.

5. The compound as claimed in claim 2,
wherein
Z2 consists of a peptide or peptide-based moiety having the Formula II:
(SEQ ID NO: 1)
X1-X2-X3-X4 (II)
wherein
X1 and X2 are independently S or Hse;
X3 is M or Nle; and
X4 is P.

6. The compound as claimed in claim 2, wherein Z2 has the amino acid sequence:
(SEQ ID NO: 3)
SSMP or
(SEQ ID NO: 4)
SS(Nle)P.

7. The compound as claimed in claim 2, wherein Z2 consists of a peptide and wherein the amino acid sequence of the peptide is
(SEQ ID NO: 5)
X5-X6-X7-X8
wherein
X5 is E or D
X6 is D or E
X7 is D or E, and
X8 is I or L.

8. The compound as claimed in claim 7, wherein Z2 consists of a peptide, wherein the amino acid sequence of the peptide is selected from the group consisting of:
(SEQ ID NO: 6)
DDDI,
(SEQ ID NO: 7)
EEEI,
(SEQ ID NO: 8)
MDEL,
(SEQ ID NO: 9)
DEDI,
(SEQ ID NO: 10)
DEEL,
(SEQ ID NO: 11)
EDDI,
(SEQ ID NO: 12)
EDEI,
(SEQ ID NO: 13)
EEDL,
(SEQ ID NO: 14)
EEEL,
(SEQ ID NO: 15)
DDEI,
(SEQ ID NO: 16)
EDQL,
(SEQ ID NO: 17)
ESEL,
(SEQ ID NO: 18)
DEEI,
(SEQ ID NO: 19)
EEDI,
(SEQ ID NO: 20)
EDEL, and
(SEQ ID NO: 21)
QEEI.

9. The compound as claimed in claim 8, wherein the amino acid sequence of the peptide is selected from the group consisting of:
(SEQ ID NO: 6)
DDDI,
(SEQ ID NO: 7)
EEEI,
(SEQ ID NO: 11)
EDDI,
(SEQ ID NO: 19)
EEDI, and
(SEQ ID NO: 20)
EDEL.

10. The compound as claimed in claim 2, wherein Z3 is K or is absent.

11. The compound as claimed in claim 2, wherein Z4 is —NH₂ or N-alkyl.

12. The compound as claimed in claim 2, which additionally comprises a targeting moiety.

13. The compound as claimed in claim 12, wherein the targeting moiety is a cancer cell-targeting moiety.

14. The peptidomimetic of a compound as claimed in claim 2.

15. A pharmaceutical composition comprising a compound as claimed in claim 2, optionally together with one or more diluents, carriers or excipients.

16. (canceled)

17. A method of treating or preventing a disease or disorder associated with NatA activity, the method comprising administering an effective amount of a compound as claimed in claim 2, wherein Z2 has the sequence of SEQ ID NO: 1, to a subject in need thereof.

18-19. (canceled)

20. A method of treating or preventing cancer, the method comprising administering an effective amount of a compound as claimed in claim 2, wherein Z2 has the sequence of SEQ ID NO: 1, to a subject in need thereof.

21-22. (canceled)

23. The 4-method as claimed in claim 20, wherein the cancer is selected from the group consisting of
lymphomas, leukaemias, neuroblastomas, glioblastomas, carcinomas, adenocarcinomas, melanomas, lung cancer, breast cancer, hepatocellular carcinoma, colorectal cancer, pancreatic cancer, ovarian cancer, gastric cancer, non-small cell lung cancer, papillary thyroid carcinoma, neuroblastoma, prostate cancer and thyroid cancer.

24. (canceled)

25. A method of treating or preventing a disease or disorder associated with Naa80 activity, the method comprising administering an effective amount of a compound as claimed in claim 2, wherein the amino acid sequence of Z2 is SEQ ID NO: 2, to a subject in need thereof.

26-27. (canceled)

28. A method of enhancing cell mobility or wound healing, the method comprising administering an effective amount of a compound as claimed in claim 2, wherein the amino acid sequence of Z2 is SEQ ID NO: 2, to a subject in need thereof.

29-31. (canceled)


SEQUENCE LISTING (63 sequences)

SEQ ID NO: 1 - protein, 4 aa, Artificial Sequence
  Peptide
  MISC_FEATURE (1)..(1): Xaa is S or Hse
  MISC_FEATURE (2)..(2): Xaa is S or Hse
  MISC_FEATURE (3)..(3): Xaa is M, Nle, a non-natural amino acid, a C1-5 alkyl group or non-cyclic alkyl group, or a C1-5 ether group
  MISC_FEATURE (3)..(3): Xaa is M, Nle, a non-natural amino acid, a C1-5 cyclic or non-cyclic alkyl group, or a C1-5 ether group
  MISC_FEATURE (4)..(4): Xaa is P or is absent
XXXX

SEQ ID NO: 2 - protein, 4 aa, Artificial Sequence
  Peptide
  MISC_FEATURE (1)..(1): Xaa is D, E, M, or Q
  MISC_FEATURE (2)..(2): Xaa is D, E, or S
  MISC_FEATURE (3)..(3): Xaa is D, E, or Q
  MISC_FEATURE (4)..(4): Xaa is I or L
XXXX

SEQ ID NO: 3 - protein, 4 aa, Artificial Sequence
  Peptide
SSMP

SEQ ID NO: 4 - protein, 4 aa, Artificial Sequence
  Peptide
  MISC_FEATURE (3)..(3): Xaa is Nle
SSXP

SEQ ID NO: 5 - protein, 4 aa, Artificial Sequence
  Peptide
  MISC_FEATURE (1)..(4): Xaa is E or D
  MISC_FEATURE (2)..(2): Xaa is D or E
  MISC_FEATURE (3)..(3): Xaa is D or E
  MISC_FEATURE (4)..(4): Xaa is I or L
XXXX

SEQ ID NO: 6 - protein, 4 aa, Artificial Sequence
  Peptide
DDDI

SEQ ID NO: 7 - protein, 4 aa, Artificial Sequence
  Peptide
EEEI

SEQ ID NO: 8 - protein, 4 aa, Artificial Sequence
  Peptide
MDEL

SEQ ID NO: 9 - protein, 4 aa, Artificial Sequence
  Peptide
DEDI

SEQ ID NO: 10 - protein, 4 aa, Artificial Sequence
  Peptide
DEEL

SEQ ID NO: 11 - protein, 4 aa, Artificial Sequence
  Peptide
EDDI

SEQ ID NO: 12 - protein, 4 aa, Artificial Sequence
  Peptide
EDEI

SEQ ID NO: 13 - protein, 4 aa, Artificial Sequence
  Peptide
EEDL

SEQ ID NO: 14 - protein, 4 aa, Artificial Sequence
  Peptide
EEEL

SEQ ID NO: 15 - protein, 4 aa, Artificial Sequence
  Peptide
DDEI

SEQ ID NO: 16 - protein, 4 aa, Artificial Sequence
  Peptide
EDQL

SEQ ID NO: 17 - protein, 4 aa, Artificial Sequence
  Peptide
ESEL

SEQ ID NO: 18 - protein, 4 aa, Artificial Sequence
  Peptide
DEEI

SEQ ID NO: 19 - protein, 4 aa, Artificial Sequence
  Peptide
EEDI

SEQ ID NO: 20 - protein, 4 aa, Artificial Sequence
  Peptide
EDEL

SEQ ID NO: 21 - protein, 4 aa, Artificial Sequence
  Peptide
QEEI

SEQ ID NO: 22 - protein, 10 aa, Artificial Sequence
  Peptide
  MISC_FEATURE (1)..(1): Xaa is A or T
  MISC_FEATURE (5)..(5): Xaa is V, I, or C
XALVXDNGSG

SEQ ID NO: 23 - protein, 5 aa, Artificial Sequence
  Peptide
  MISC_FEATURE (3)..(3): Xaa is Nle
SSXPK

SEQ ID NO: 24 - protein, 5 aa, Artificial Sequence
  Cyclic targeting moiety, Y is D-tyrosine
RGDYK

SEQ ID NO: 25 - protein, 7 aa, Artificial Sequence
  Cyclic CPP9 targeting moiety
  MISC_FEATURE (1)..(1): D-amino acid
  MISC_FEATURE (2)..(2): X is 2-napthylalanine
  MISC_FEATURE (4)..(4): D-amino acid
  MISC_FEATURE (6)..(6): D-amino acid
FXRRRRQ

SEQ ID NO: 26 - protein, 4 aa, Artificial Sequence
  CoA-Ac-peptide
SESS

SEQ ID NO: 27 - protein, 4 aa, Artificial Sequence
  CoA-Ac-peptide
SASE

SEQ ID NO: 28 - protein, 4 aa, Artificial Sequence
  CoA-Ac-peptide
AASE

SEQ ID NO: 29 - protein, 4 aa, Artificial Sequence
  CoA-Ac-peptide
SSSE

SEQ ID NO: 30 - protein, 4 aa, Artificial Sequence
  CoA-Ac-peptide
SAAE

SEQ ID NO: 31 - protein, 4 aa, Artificial Sequence
  CoA-Ac-peptide
SSAE

SEQ ID NO: 32 - protein, 4 aa, Artificial Sequence
  CoA-Ac-peptide
SYAE

SEQ ID NO: 33 - protein, 4 aa, Artificial Sequence
  CoA-Ac-peptide
SSAA

SEQ ID NO: 34 - protein, 4 aa, Artificial Sequence
  CoA-Ac-peptide
SSME

SEQ ID NO: 35 - protein, 5 aa, Artificial Sequence
  CoA-Ac-peptide
SSMPV

SEQ ID NO: 36 - protein, 4 aa, Artificial Sequence
  CoA-Ac-peptide
  MISC_FEATURE (3)..(3): Xaa is Nle
PSXP

SEQ ID NO: 37 - DNA, 20 bp, Artificial Sequence
  NAA80 sense primer
caacatgcaagagctgactc

SEQ ID NO: 38 - DNA, 25 bp, Artificial Sequence
  NAA80 antisense primer
gatgtctttttccatccagaatatg

SEQ ID NO: 39 - protein, 17 aa, Artificial Sequence
  C-terminal sequence
RWGRPVGRRRRPVRVYP

SEQ ID NO: 40 - protein, 7 aa, Artificial Sequence
  EEEI-24 peptide
EEEIAAL

SEQ ID NO: 41 - protein, 7 aa, Artificial Sequence
  DDDI-24 peptide
DDDIAAL

SEQ ID NO: 42 - protein, 7 aa, Artificial Sequence
  MDDD-24 peptide
MDDDIAA

SEQ ID NO: 43 - protein, 7 aa, Artificial Sequence
  SESS-24 peptide
SESSSKS

SEQ ID NO: 44 - protein, 7 aa, Artificial Sequence
  AVFA-24 peptide
AVFADLD

SEQ ID NO: 45 - protein, 7 aa, Artificial Sequence
  MDEL-24 peptide
MDELFPL

SEQ ID NO: 46 - protein, 7 aa, Artificial Sequence
  MELL-24 peptide
MELLSPP

SEQ ID NO: 47 - protein, 7 aa, Artificial Sequence
  MLGT-24 peptide
MLGTGPA

SEQ ID NO: 48 - protein, 7 aa, Artificial Sequence
  MTNK-24 peptide
MTNKSSL

SEQ ID NO: 49 - protein, 7 aa, Artificial Sequence
  MLGP-24 peptide
MLGPEGG

SEQ ID NO: 50 - protein, 7 aa, Artificial Sequence
  MAPL-24 peptide
MAPLDLD

SEQ ID NO: 51 - protein, 7 aa, Artificial Sequence
  MKKS-24 peptide
MKKSYSG

SEQ ID NO: 52 - protein, 4 aa, Artificial Sequence
  Negative control peptide
MLGT

SEQ ID NO: 53 - protein, 4 aa, Artificial Sequence
  Inhibitor peptide
PDEL

SEQ ID NO: 54 - protein, 4 aa, Artificial Sequence
  NatB peptide
MELL

SEQ ID NO: 55 - protein, 4 aa, Artificial Sequence
  NatD peptide
SGRG

SEQ ID NO: 56 - protein, 4 aa, Artificial Sequence
  NatE peptide
MLGP

SEQ ID NO: 57 - protein, 4 aa, Artificial Sequence
  NatF peptide
MAPL

SEQ ID NO: 58 - protein, 61 aa, Artificial Sequence
  Beta (cyto)-actin
MDDDIAALVVDNGSGMCKAGFAGDDAPRAVFPSIVGRPRHQGVMVGMGQKDSYVGDEAQSK

SEQ ID NO: 59 - protein, 61 aa, Artificial Sequence
  Gamma (cyto)-actin
MEEEIAALVIDNGSGMCKAGFAGDDAPRAVFPSIVGRPRHQGVMVGMGQKDSYVGDEAQSK

SEQ ID NO: 60 - protein, 63 aa, Artificial Sequence
  Alpha (smooth)-actin
MCEEEDSTALVCDNGSGLCKAGFAGDDAPRAVFPSIVGRPRHQGVMVGMGQKDSYVGDEAQSK

SEQ ID NO: 61 - protein, 62 aa, Artificial Sequence
  Gamma (smooth)-actin
MCEEETTALVCDNGSGLCKAGFAGDDAPRAVFPSIVGRPRHQGVMVGMGQKDSYVGDEAQSK

SEQ ID NO: 62 - protein, 63 aa, Artificial Sequence
  Alpha (cardiac)-actin
MCDDEETTALVCDNGSGLVKAGFAGDDAPRAVFPSIVGRPRHQGVMVGMGQKDSYVGDEAQSK

SEQ ID NO: 63 - protein, 63 aa, Artificial Sequence
  Alpha (skeletal)-actin
MCDEDETTALVCDNGSGLVKAGFAGDDAPRAVFPSIVGRPRHQGVMVGMGQKDSYVGDEAQSK